SEQUENCE LISTING (3 sequences)

SEQ ID NO: 1 - protein, 775 aa, Homo sapiens
MASAGHIITLLLWGYLLELWTGGHTADTTHPRLRLSHKELLNLNRTSIFHSPFGFLDLHTMLLDEYQERLFVGGRDLVYSLSLERISDGYKEIHWPSTALKMEECIMKGKDAGECANYVRVLHHYNRTHLLTCGTGAFDPVCAFIRVGYHLEDPLFHLESPRSERGRGRCPFDPSSSFISTLIGSELFAGLYSDYWSRDAAIFRSMGRLAHIRTEHDDERLLKEPKFVGSYMIPDNEDRDDNKVYFFFTEKALEAENNAHAIYTRVGRLCVNDVGGQRILVNKWSTFLKARLVCSVPGMNGIDTYFDELEDVFLLPTRDHKNPVIFGLFNTTSNIFRGHAICVYHMSSIRAAFNGPYAHKEGPEYHWSVYEGKVPYPRPGSCASKVNGGRYGTTKDYPDDAIRFARSHPLMYQAIKPAHKKPILVKTDGKYNLKQIAVDRVEAEDGQYDVLFIGTDNGIVLKVITIYNQEMESMEEVILEELQIFKDPVPIISMEISSKRQQLYIGSASAVAQVRFHHCDMYGSACADCCLARDPYCAWDGISCSRYYPTGTHAKRRFRRQDVRHGNAAQQCFGQQFVGDALDKTEEHLAYGIENNSTLLECTPRSLQAKVIWFVQKGRETRKEEVKTDDRVVKMDLGLLFLRLHKSDAGTYFCQTVEHSFVHTVRKITLEVVEEEKVEDMFNKDDEEDRHHRMPCPAQSSISQGAKPWYKEFLQLIGYSNFQRVEEYCEKVWCTDRKRKKLKMSPSKWKYANPQEKKLRSKPEHYRLPRHTLDS

SEQ ID NO: 2 - protein, 4 aa, Artificial sequence
  sequence is synthesized
GRAG

SEQ ID NO: 3 - protein, 4 aa, Artificial sequence
  sequence is synthesized
GGGS

(19) United States
(12) Patent Application Publication
(10) Pub. No.: US 2009/0181035 A1
(43) Pub. Date: Jul. 16, 2009

(54) PLEXIND1 AGONISTS AND THEIR USE

(75) Inventors: Ryan J. Watts, San Mateo, CA (US); Alexander W. Koch, Millbrae, CA (US); Marc Tessier-Lavigne, Woodside, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/351,685

(22) Filed: Jan. 9, 2009

Related U.S. Application Data
(60) Provisional application No. 61/020,296, filed on Jan. 10, 2008.

(51) Int. Cl. A61K 39/395; A61K 38/02; C07K 16/00; C07K 14/47; A61P 35/00; C07H 21/00; C12N 15/63

(52) U.S. Cl. 424/158.1; 424/130.1; 514/12; 530/387.1; 530/300; 530/350; 536/23.53; 435/320.1

(57) ABSTRACT

The present invention relates to PLEXIND1 agonists and their use for the treatment of disorders associated with angiogenesis, including cancer.

### CROSS REFERENCE TO RELATED APPLICATIONS

This application claims the benefit of U.S. Provisional Application No. 61/020,296, filed 10 Jan. 2008, the disclosure of which is incorporated herein by reference in its entirety.

### FIELD OF THE INVENTION

The present invention concerns methods and compositions relating to the use of PlexinD1 agonists for the treatment of disorders associated with angiogenesis.

### BACKGROUND OF THE INVENTION

It is now well established that angiogenesis is an important contributor to the pathogenesis of a variety of disorders. These include solid tumors and metastasis, atherosclerosis, retrolental fibroplasia, hemangiomas, chronic inflammation, intraocular neovascular diseases such as proliferative retinopathies, e.g., diabetic retinopathy, retinal vein occlusion (RVO), age-related macular degeneration (AMD), neovascular glaucoma, immune rejection of transplanted corneal tissue and other tissues, rheumatoid arthritis, and psoriasis. Duda et al., 2007, J. Clin. Oncology 25(26): 4033-42; Kesisis et al., 2007, Curr. Pharm. Des. 13: 2795-809; Zhang & Ma, 2007, Prog. Ret. & Eye Res. 26: 1-37.

In the case of tumor growth, angiogenesis allows the tumor cells to acquire a growth advantage and proliferative autonomy compared to the normal cells. A tumor usually begins as a single aberrant cell which is able to grow only to a size of a few cubic millimeters due to the distance from available capillary beds and it can stay ‘dormant’ without further growth or dissemination for a long period of time. Some tumor cells then switch to the angiogenic phenotype to activate endothelial cells, which proliferate and mature into new capillary blood vessels. These newly formed blood vessels not only allow for continued growth of the primary tumor, but also for the dissemination and recolonization of metastatic tumor cells. The mechanisms that control the angiogenic switch are not well understood, but it is believed that neovascularization of tumor mass results from the net balance of a multitude of angiogenesis stimulators and inhibitors.

Semaphorins are a family of secreted and membrane bound proteins that comprise a shared extracellular domain (the “sema” domain). Semaphorins play a role in angiogenesis as well as in the immune response and in the nervous system acting through multiple receptors, including several designated plexins (Kolodkin et al. 1993, Cell 75: 1389-99; Hall et al., 1996, Proc. Natl. Acad. Sci. USA 93: 11780-85; Miao et al., 2000, FASEB J. 14: 2532-9; Tamagnone et al., 1999, Cell 99: 71-80). Both the semaphorins and plexins are expressed in tumor cells (Christensen et al., 1998, Cancer Res. 58: 1238-44; Brambilla et al., 2000, Am. J. Pathol. 56: 939-50; Trusolino and Comoglio, 2002, Nature Rev. Cancer 2: 289-300).

The various roles played by semaphorins appear to be cell- and tissue-specific and can sometimes appear contradictory. For example, Sema3E is a repulsive cue for sensory and sympathetic neurons, but a chemoattractant and neurite outgrowth inducer for PC12 cells (Chen et al., 1997, Neuron 19: 547-59; Miyazaki et al., 1999, Neuroscience 93: 401-08; Sakai et al., 1999, J. Biol. Chem. 274: 29666-71). In addition, Sema3E and PlexinD1 have been shown to be involved in vascular development and patterning (Gitler et al. 2004, Dev. Cell 7: 107-16; Torres-Vasquez et al. 2004, Dev. Cell 7: 117-23; Gu et al. 2005, Science 307: 265-68). Similarly, some of the semaphorins are believed to act as tumor suppressors (SEMA3B and SEMA3F; Xiang et al., 1996, Genomics 32: 39-48; Sekido et al., 1996, Proc. Nat. Acad. Sci. USA 93: 4120-25; Brambilla et al., 2000, Am. J. Pathol. 56: 939-50; Tomizawa et al., 2001, Proc. Natl. Acad. Sci. USA 98: 13954-59) whereas others are thought to promote tumor progression (SEMA3C and SEMA3E; Martin-Satue and Blanco 1999, J. Surg. Oncol. 72: 18-23; Yamada et al. 1997, Proc. Natl. Acad. Sci. USA 94: 14713-18; Christensen et al., 1998, Cancer Res. 58: 1238-44). Another specific example is Sema4A, which has been shown to suppress angiogenesis and is proposed as a therapeutic agent for cancer (Toyofuku et al. 2007, EMBO J. 26: 1373-84). In contrast, it has been proposed that inhibiting SEMA3E expression, inhibiting cleavage of its extracellular domain or interfering with its interaction with or ability to activate Plexin receptors may be useful for the treatment of cancer (see, e.g., US Patent Application US 2007/0010433A1).

### SUMMARY OF THE INVENTION

The present invention is based, in part, on the surprising discovery that agonizing PlexinD1 activity inhibits angiogenesis, including cancer-associated angiogenesis.

In one aspect, the invention provides a method for treating a proliferative disorder in an animal comprising administering to the animal a polypeptide comprising a fragment of Sema3E, wherein said polypeptide is a PlexinD1 agonist. In some embodiments, the proliferative disorder is cancer. In some embodiments, the cancer is carcinoma, lymphoma, blastoma, sarcoma, leukemia, squamous cell cancer, lung cancer, brain cancer, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, colorectal cancer, endometrial, uterine carcinoma, salivary gland carcinoma, renal cancer, prostate cancer, vulval cancer, thyroid cancer, hepatic carcinoma or cancers of the head and neck. In some embodiments, the fragment of Sema3E lacks the immunoglobulin-like domain, the first furin cleavage site and/or does not bind to neuropilin-1 (Nrp1). In some embodiments, the method further comprises administering a chemotherapeutic agent and/or a VEGF antagonist. In some embodiments, the VEGF antagonist is selected from the group consisting of an antisense RNA, an siRNA, an aptamer, a polypeptide comprising a VEGF-binding fragment of a VEGF receptor, and an anti-VEGF antibody (e.g. Avastin®).

In another aspect, the invention provides a polypeptide comprising a fragment of Sema3E, wherein said polypeptide is a PlexinD1 agonist and wherein said fragment lacks the first furin cleavage site. In some embodiments, the fragment of Sema3E further lacks the immunoglobulin-like domain. In some embodiments, the polypeptide also comprises the Fc portion of an immunoglobulin (e.g. IgG1). In some embodiments, the polypeptide comprises a linker between said fragment of Sema3E and said Fc portion of an immunoglobulin (e.g. GRAG (SEQ ID NO: 2) or GGGS (SEQ ID NO: 3)). In some embodiments, the fragment of Sema3E comprises from any one of amino acids 1-32 of SEQ ID NO: 1 to any one of amino acids 516-555 of SEQ ID NO: 1, e.g. amino acids 1-554 or 26-555 of SEQ ID NO: 1. In some embodiments, the invention provides a nucleic acid encoding a polypeptide of the invention, a vector comprising the nucleic acid and in vitro host cells comprising the vectors or nucleic acids.

In some embodiments, the invention provides a method for inhibiting angiogenesis in an animal comprising administering to the animal a polypeptide of the invention. In some embodiments, a second angiogenesis inhibitor is also administered to the animal. In some embodiments, the second angiogenesis inhibitor is a VEGF antagonist, e.g. an antisense RNA, an siRNA, an aptamer, a polypeptide comprising a VEGF-binding fragment of a VEGF receptor, and an anti-VEGF antibody (e.g. Avastin®).

In some embodiments, the invention provides a method for treating cancer in an animal comprising administering to the animal a polypeptide of the invention. In some embodiments, the cancer is carcinoma, lymphoma, blastoma, sarcoma, leukemia, squamous cell cancer, lung cancer, brain cancer, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, colorectal cancer, endometrial, uterine carcinoma, salivary gland carcinoma, renal cancer, prostate cancer, vulval cancer, thyroid cancer, hepatic carcinoma or cancers of the head and neck. In some embodiments, a second angiogenesis inhibitor is also administered to the animal. In some embodiments, the second angiogenesis inhibitor is a VEGF antagonist, e.g. an antisense RNA, an siRNA, an aptamer, a polypeptide comprising a VEGF-binding fragment of a VEGF receptor, and an anti-VEGF antibody (e.g. Avastin®)

### BRIEF DESCRIPTION OF THE DRAWINGS

FIG. 1 shows Biacore binding data for PlexinD1 binding to a soluble Sema3E sema domain (Sema3E^{SD}) construct.

FIG. 2 shows that His-tagged Sema3E^{SD} inhibits HUVEC migration induced by either HGF or VEGF (A) and that a Sema3E^{SD}-Fc immunoadhesin inhibits VEGF-induced HUVEC migration and does not by itself stimulate migration (B).

FIG. 3 shows that Sema3E^{SD} inhibits VEGF-induced HUVEC sprout formation.

FIG. 4 shows that Sema3E^{SD}-mediated inhibition of HUVEC migration it blocked by a soluble, extracellular fragment of PlexinD1 (PlxD1-Fc).

FIG. 5 shows that several anti-PlexinD1 antibodies block the ability of Sema3E^{SD} to inhibit HUVEC migration.

FIG. 6 shows that Sema3E^{SD}-Fc inhibits tumor growth in vivo in a mouse xenograft model.

### DETAILED DESCRIPTION OF THE PREFERRED EMBODIMENTS

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

All references cited herein, including patent applications and publications, are incorporated by reference in their entirety.

### DEFINITIONS

The terms “SEMA3E” or “Sema3E”, as used herein, refer, unless specifically or contextually indicated otherwise, to any native or variant (whether natural or synthetic) SEMA3E polypeptide. SEMA3E consists of the following domains (with approximate amino-acid endpoints identified according to the sequence in SEQ ID NO: 1): SEMA domain (26-516); first furin cleavage site (555-560), plexin/semaphorin/integrin (PSI) domain (519-572); immunoglobulin-like domain (581-669); second furin cleavage site (737-741); basic C-terminal tail (737-770). The term “native sequence” specifically encompasses all naturally occurring forms, including naturally occurring variant forms (e.g., alternatively spliced forms) and naturally occurring allelic variants. The term “wild-type SEMA3E” generally refers to a polypeptide comprising the amino acid sequence of a naturally occurring SEMA3E protein. The term “wild-type SEMA3E sequence” generally refers to an amino acid sequence found in a naturally occurring SEMA3E. GenBank® accession numbers for representative SEMA3E polypeptide sequences are: AC004954, AC079799, AC079987 (human), and BC057956 (mouse). The sequences of a human wild-type SEMA3E is also shown below:

| (SEQ ID NO: 1) | |
|---|---|
| MASAGHIITLLLWGYLLELWTGGHTADTTHPRLRLSHKELLNLNRTSIFH | 50 |
| SPFGFLDLHTMLLDEYQERLFVGGRDLVYSLSLERISDGYKEIHWPSTAL | 100 |
| KMEECIMKGKDAGECANYVRVLHHYNRTHLLTCGTGAFDPVCAFIRVGYH | 150 |
| LEDPLFHLESPRSERGRGRCPFDPSSSFISTLIGSELFAGLYSDYWSRDA | 200 |
| AIFRSMGRLAHIRTEHDDERLLKEPKFVGSYMIPDNEDRDDNKVYFFFTE | 250 |
| KALEAENNAHAIYTRVGRLCVNDVGGQRILVNKWSTFLKARLVCSVPGMN | 300 |
| GIDTYFDELEDVFLLPTRDHKNPVIFGLFNTTSNIFRGHAICVYHMSSIR | 350 |
| AAFNGPYAHKEGPEYHWSVYEGKVPYPRPGSCASKVNGGRYGTTKDYPDD | 400 |
| AIRFARSHPLMYQAIKPAHKKPILVKTDGKYNLKQIAVDRVEAEDGQYDV | 450 |
| LFIGTDNGIVLKVITIYNQEMESMEEVILEELQIFKDPVPIISMEISSKR | 500 |
| QQLYIGSASAVAQVRFHHCDMYGSACADCCLARDPYCAWDGISCSRYYPT | 550 |
| GTHAKRRFRRQDVRHGNAAQQCFGQQFVGDALDKTEEHLAYGIENNSTLL | 600 |
| ECTPRSLQAKVIWFVQKGRETRKEEVKTDDRVVKMDLGLLFLRLHKSDAG | 650 |
| TYFCQTVEHSFVHTVRKITLEVVEEEKVEDMFNKDDEEDRHHRMPCPAQS | 700 |
| SISQGAKPWYKEFLQLIGYSNFQRVEEYCEKVWCTDRKRKKLKMSPSKWK | 750 |
| YANPQEKKLRSKPEHYRLPRHTLDS. | |

The term “SEMA3E^{SD}” refers to that portion of SEMA3E that corresponds essentially to the extracellular sema domain. For the wild-type human SEMA3E shown as SEQ ID NO: 1 above, the sema domain corresponds to a sequence with an amino terminus that begins at any one of amino acids 1-32 and that ends at any one of amino acids 516-560.

A “chimeric SEMA3E” molecule is a polypeptide comprising full-length SEMA3E or one or more domains (e.g., the sema domain) thereof fused or bonded to heterologous polypeptide. Sometimes when the chimeric SEMA3E molecule comprises only a particular domain, that domain is identified, e.g. a “chimeric SEMA3E^{SD}” molecule. The chimeric SEMA3E molecule will generally share at least one biological property in common with naturally occurring SEMA3E. An example of a chimeric SEMA3E molecule is one that is epitope tagged for purification purposes. Another chimeric SEMA3E molecule is an SEMA3E immunoadhesin.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG1, IgG2, IgG3, or IgG4 subtypes, IgA (including IgA1 and IgA2), IgE, IgD or IgM.

The term “SEMA3E immunoadhesin” refers to a chimeric SEMA3E molecule that also comprises an immunoglobulin sequence. In some instances the SEMA3E immunoadhesin comprises SEMA3E^{SD} or a fragment thereof sufficient to agonize PLEXIND1 activity. The immunoglobulin sequence preferably, but not necessarily, is an immunoglobulin constant domain (Fc region). Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use. In some embodiments, the Fc region is a native sequence Fc region. In some embodiments, the Fc region is a variant Fc region. In some embodiments, the Fc region is a functional Fc region.

The term “chimeric heteroadhesin” refers to a complex of chimeric molecules (amino acid sequences) in which each chimeric molecule combines a biologically active portion, such as the extracellular domain of each of the heteromultimeric receptor monomers, with a multimerization domain. The “multimerization domain” promotes stable interaction of the chimeric molecules within the heteromultimer complex. The multimerization domains may interact via an immunoglobulin sequence, leucine zipper, a hydrophobic region, a hydrophilic region, or a free thiol that forms an intermolecular disulfide bond between the chimeric molecules of the chimeric heteromultimer. The multimerization domain may comprise an immunoglobulin constant region. In addition a multimerization region may be engineered such that steric interactions not only promote stable interaction, but further promote the formation of heterodimers over homodimers from a mixture of monomers. “Protuberances” are constructed by replacing small amino acid side chains from the interface of the first polypeptide with larger side chains (e.g. tyrosine or tryptophan). Compensatory “cavities” of identical or similar size to the protuberances are optionally created on the interface of the second polypeptide by replacing large amino acid side chains with smaller ones (e.g. alanine or threonine). The immunoglobulin sequence preferably, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but preferably IgG1 or IgG3.

An “isolated” polypeptide is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated polypeptide includes the polypeptide in situ within recombinant cells since at least one component of the polypeptide's natural environment will not be present. Ordinarily, however, isolated polypeptide will be prepared by at least one purification step.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact immunoadhesins or antibodies may comprise antibody populations with all K447 residues removed, with no K447 residues removed, or a mixture with and without the K447 residue.

Unless indicated otherwise, the numbering used herein for the residues in an immunoglobulin heavy chain is that of the EU index as in Kabat et al., Sequences of Proteins of Immunological Interest, 5th Ed. Public Health Service, National Institutes of Health, Bethesda, Md. (1991). The “EU index as in Kabat” refers to the residue numbering of the human IgG1 EU antibody.

A “functional Fc region” possesses at least one “effector function” of a native sequence Fc region. Exemplary “effector functions” include C1q binding; complement dependent cytotoxicity; Fc receptor binding; antibody-dependent cell-mediated cytotoxicity (ADCC); phagocytosis; and down regulation of cell surface receptors (e.g. B cell receptor; BCR). Such effector functions generally require the Fc region to be combined with a binding domain and can be assessed using various assays known in the art or disclosed herein.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith. A variant Fc region may be chosen, e.g., to alter antibody half-life in serum or to modify or eliminate an effector function.

The terms “antibody” and “immunoglobulin” are used interchangeably in the broadest sense and include monoclonal antibodies (for e.g., full length or intact monoclonal antibodies), polyclonal antibodies, multivalent antibodies, multispecific antibodies (e.g., bispecific antibodies so long as they exhibit the desired biological activity) and may also include certain antibody fragments (as described in greater detail herein). An antibody can be human, humanized and/or affinity matured.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method and technologies for producing human or human-like antibodies in animals that have parts or all of the human immunoglobulin loci or genes encoding human immunoglobulin sequences.

An “antigen” is a predetermined antigen to which an antibody can selectively bind. The target antigen may be polypeptide, carbohydrate, nucleic acid, lipid, hapten or other naturally occurring or synthetic compound. Preferably, the target antigen is a polypeptide.

An “agonist” antibody is one which activates or increases biological activity of the antigen it binds, e.g. by increasing signaling by a receptor. An agonist antibody may be generated by methods described herein.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

A “disorder” or “disease” is any condition that would benefit from treatment with a substance/molecule or method of the invention, e.g. disorders or diseases associated with angiogenesis. This includes chronic and acute disorders or diseases including those pathological conditions which predispose the mammal to the disorder in question. Non-limiting examples of disorders to be treated herein include cancer and non-neoplastic disorders.

The terms “cell proliferative disorder” and “proliferative disorder” refer to disorders that are associated with some degree of abnormal cell proliferation. In some embodiments, the cell proliferative disorder is cancer.

“Tumor”, as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, gastric cancer, melanoma, and various types of head and neck cancer. Dysregulation of angiogenesis can lead to many disorders that can be treated by compositions and methods of the invention. These disorders include both non-neoplastic and neoplastic conditions. Neoplastic disorders include but are not limited to those described above.

Non-neoplastic disorders include but are not limited to undesired or aberrant hypertrophy, arthritis, rheumatoid arthritis (RA), psoriasis, psoriatic plaques, sarcoidosis, atherosclerosis, atherosclerotic plaques, diabetic and other proliferative retinopathies including retinopathy of prematurity, retinal vein occlusion (RVO) and central retinal vein occlusion (CRVO), retrolental fibroplasia, choroidal neovascularization, neovascular glaucoma, age-related macular degeneration (AMD), diabetic macular edema, corneal neovascularization, corneal graft neovascularization, corneal graft rejection, retinal/choroidal neovascularization, neovascularization of the angle (rubeosis), ocular neovascular disease, pathological myopia, von Hippel-Lindau disease, histoplasmosis of the eye, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertrophic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), hemophilic joints, hypertrophic scars, inhibition of hair growth, Osler-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies are used to delay development of a disease or disorder.

A “subject” is a vertebrate, preferably a mammal, more preferably a human. Subjects include, but are not limited to, farm animals (such as cows and sheep), sport animals, pets (such as cats, dogs and horses), primates, mice and rats.

A “therapeutically effective amount” refers to an amount effective, at dosages and for periods of time necessary, to achieve a desired therapeutic result. The necessary amount of a substance/molecule may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re⁸⁶, Re¹⁸⁸, Sm⁵³, Bi²¹², P³² and radioactive isotopes of Lu), chemotherapeutic agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed herein.

A “chemotherapeutic agent” is a chemical compound useful in the treatment of cancer. Non-limiting examples of chemotherapeutic agents include alkylating agents; alkyl sulfonates; aziridines; ethylenimines and methylamelamines; acetogenins; camptothecins; nitrosureas; antibiotics; anti-metabolites; folic acid analogues; purine and pyrimidine analogs; anti-adrenals; folic acid replenishers; maytansinoids; trichothecenes; taxoids; platinum analogs; pharmaceutically acceptable salts, acids or derivatives of any of the above; as well as combinations of two or more of the above. Also included in this definition are anti-hormonal agents that act to regulate, reduce, block, or inhibit the effects of hormones that can promote the growth of cancer, and are often in the form of systemic, or whole-body treatment. They may be hormones themselves.

The “pathology” of a disease includes all phenomena that compromise the well-being of the patient. For cancer, this includes, without limitation, abnormal or uncontrollable cell growth, metastasis, interference with the normal functioning of neighboring cells, release of cytokines or other secretory products at abnormal levels, suppression or aggravation of inflammatory or immunological response, etc.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The terms “VEGF” and “VEGF-A” are used interchangeably to refer to the 165-amino acid vascular endothelial cell growth factor and related 121-, 145-, 183-, 189-, and 206-amino acid vascular endothelial cell growth factors, as described by, e.g., Robinson & Stringer, 2001, J. Cell Sci., 144(5): 853-65, together with the naturally occurring allelic and processed forms thereof.

A “VEGF antagonist” refers to a molecule capable of neutralizing, blocking, inhibiting, abrogating, reducing or interfering with a VEGF activity including its binding to one or more VEGF receptors. VEGF antagonists include anti-VEGF antibodies and antigen-binding fragments thereof, receptor molecules and derivatives which bind specifically to VEGF thereby sequestering its binding to one or more receptors, anti-VEGF receptor antibodies and VEGF receptor antagonists such as small molecule inhibitors of the VEGFR tyrosine kinases, and fusions proteins, e.g., VEGF-Trap (Regeneron), VEGF121-gelonin (Peregrine). VEGF antagonists also include antagonist variants of VEGF, antisense molecules directed to VEGF, RNA aptamers, and ribozymes against VEGF or VEGF receptors.

An anti-VEGF antibody can be used as a therapeutic agent in targeting and interfering with diseases or conditions wherein the VEGF activity is involved. Such an anti-VEGF antibody will usually not exhibit significant binding to other proteins related to VEGF such as VEGF-B or VEGF-C, nor other growth factors such as P1GF, PDGF or bFGF. The anti-VEGF antibody “Bevacizumab (BV)”, also known as “rhuMAb VEGF” or “Avastin®”, is a recombinant humanized anti-VEGF monoclonal antibody generated according to Presta et al., 1997, Cancer Res. 57: 4593-99. Bevacizumab and other humanized anti-VEGF antibodies, including the anti-VEGF antibody fragment “ranibizumab”, also known as “Lucentis®”, are further described in U.S. Pat. No. 6,884,879.

A “PLEXIND1 agonist” refers to a molecule capable of inhibiting PLEXIND1-mediated endothelial cell migration. PLEXIND1 agonists include agonist antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Agonists also include small molecule activators of PLEXIND1, and fusion proteins (including immunoadhesins), receptor molecules and derivatives which bind specifically to PLEXIND1 and agonize its activity. In some embodiments, the PLEXIND1 agonist in polypeptide that comprises a fragment of SEMA3E, e.g. SEMA3E^{SD}. In some embodiments, the PLEXIND1 agonist is an antibody.

An “anti-angiogenesis agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. For example, an anti-angiogenesis agent is an antibody or other antagonist to an angiogenic agent as defined above, e.g., antibodies to VEGF, antibodies to VEGF receptors, small molecules that block VEGF receptor signaling (e.g., PTK787/ZK2284, SU6668, SUTENT®/SU11248 (sunitinib malate), AMG706, or those described in, e.g., WO 2004/113304). Anti-angiogenesis agents also include native angiogenesis inhibitors, e.g., angiostatin, endostatin, etc. See, e.g., Klagsbrun and D'Amore, 1991, Annu. Rev. Physiol., 53: 217-39; Streit and Detmar, 2003, Oncogene, 22: 3172-79 (e.g., Table 3 listing anti-angiogenic therapy in malignant melanoma); Ferrara & Alitalo, 1999, Nature Medicine 5(12): 1359-64; Tonini et al., 2003, Oncogene, 22: 6549-56 (e.g., Table 2 listing antiangiogenic factors); and, Sato, 2003, Int. J. Clin. Oncol., 8: 200-06 (e.g., Table 1 lists Anti-angiogenic agents used in clinical trials).

Methods and Compositions of the Invention

The present invention is based in part on the discovery that agents that agonize PLEXIND1 activity are capable of inhibiting angiogenesis. In addition, such agents were found to be able to inhibit tumor growth in vivo. Accordingly, PLEXIND1 agonists are useful for pathological conditions and disorders associated with angiogenesis.

PLEXIND1 agonists can be used to treat or prevent various pathological conditions or disorders. The invention encompasses a method of inhibiting angiogenesis using an effective amount of a PLEXIND1 agonist such as, without limitation, a SEMA3E immunoadhesin or anti-PLEXIND1 agonist antibody, to activate the PLEXIND1 pathway. In another aspect the invention provides a method of treating a disorder or diseases associated with angiogenesis comprising administering an effective amount of a PLEXIND1 agonist to a subject in need of such treatment.

Combination Therapies

As indicated above, the invention provides combined therapies in which a PLEXIND1 agonist (such as a SEMA3E immunoadhesin or anti-PLEXIND1 agonist antibody) is administered with another therapy. For example, a PLEXIND1 agonist is used in combination with a chemotherapeutic agent or another anti-angiogenesis agent (including, e.g. a VEGF antagonist) to treat various neoplastic or non-neoplastic conditions. The administration of the PLEXIND1 agonist and the other therapeutic agent can be done simultaneously, e.g., as a single composition or as two or more distinct compositions using the same or different administration routes. Alternatively, or additionally, the administration can be done sequentially, in any order. Alternatively, or additionally, the steps can be performed as a combination of both sequentially and simultaneously, in any order. In some embodiments, intervals ranging from minutes to days, to weeks to months, can be present between the administrations of the two or more compositions.

The effective amounts of therapeutic agents administered will be at the physician's or veterinarian's discretion. Dosage administration and adjustment is done to achieve maximal management of the conditions to be treated. The dose will additionally depend on such factors as the type of therapeutic agent to be used and the specific patient being treated.

Production of an Immunoadhesin

The description below relates primarily to production of immunoadhesin by culturing cells transformed or transfected with a vector containing immunoadhesin nucleic acid. It is, of course, contemplated that alternative methods, which are well known in the art, may be employed to prepare immunoadhesins. For instance, the immunoadhesin sequence, or portions thereof, may be produced by direct peptide synthesis using solid-phase techniques. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the immunoadhesin may be chemically synthesized separately and combined using chemical or enzymatic methods to produce the full-length immunoadhesin.

An immunoadhesin or a chimeric heteroadhesin of the invention is preferably produced by expression in a host cell and isolated therefrom. A host cell is generally transformed with the nucleic acid of the invention. Preferably the nucleic acid is incorporated into an expression vector. Suitable host cells for cloning or expressing the vectors herein are prokaryotic host cells (such as E. coli, strains of Bacillus, Pseudomonas and other bacteria), yeast and other eukaryotic microbes, and higher eukaryote cells (such as Chinese hamster ovary (CHO) cells and other mammalian cells). The cells may also be present in live animals (for example, in cows, goats or sheep). Insect cells may also be used. Cloning and expression methodologies are well known in the art.

To obtain expression of an immunoadhesin such as the SEMA3E^{SD}-Fc molecule (described in detail in the Examples), one or more expression vector(s) is/are introduced into host cells by transformation or transfection and the resulting recombinant host cells are cultured in conventional nutrient media, modified as appropriate for inducing promoters, selecting recombinant cells, or amplifying the SEMA3E^{SD}-Fc DNA. In general, principles, protocols, and practical techniques for maximizing the productivity of in vitro mammalian cell cultures can be found in Mammalian Cell Biotechnology: a Practical Approach, M. Butler, ed. (IRL Press, 1991).

Construction of Nucleic Acid Encoding Immunoadhesin

When preparing the immunoadhesins of the present invention, preferably nucleic acid encoding an extracellular fragment of SEMA3E is fused C-terminally to nucleic acid encoding the N-terminus of an immunoglobulin constant domain sequence, however N-terminal fusions are also possible. Typically, in such fusions the encoded chimeric polypeptide will retain at least a functionally active hinge, C_{H}2 and C_{H}3 domains of the constant region of an immunoglobulin heavy chain. Fusions are also made to the C-terminus of the Fc portion of a constant domain, or immediately N-terminal to the C_{H}1 of the heavy chain or the corresponding region of the light chain. The resultant DNA fusion construct is expressed in appropriate host cells.

Nucleic acid molecules encoding amino acid sequence variants of native sequence extracellular domains (such as from SEMA3E) and/or the antibody sequences used to prepare the desired immunoadhesin, are prepared by a variety of methods known in the art. These methods include, but are not limited to, isolation from a natural source or preparation by oligonucleotide-mediated (or site-directed) mutagenesis, PCR mutagenesis, and cassette mutagenesis of an earlier prepared variant or a non-variant version of native sequence SEMA3E.

Amino acid sequence variants of native sequence extracellular domain included in the chimeric heteroadhesin are prepared by introducing appropriate nucleotide changes into the native extracellular domain DNA sequence, or by in vitro synthesis of the desired chimeric heteroadhesin monomer polypeptide. Such variants include, for example, deletions from, or insertions or substitutions of, residues in the amino acid sequence of the immunoadhesin or chimeric heteroadhesin.

In some embodiments, the nucleic acid encodes a chimeric molecule in which the SEMA3E sema domain sequence is fused to the N-terminus of the C-terminal portion of an antibody (in particular the Fc domain), containing the effector functions of an immunoglobulin, e.g. IgG1. It is possible to fuse the entire heavy chain constant region to the SEMA3E sema domain sequence. However, more preferably, a sequence beginning in the hinge region just upstream of the papain cleavage site (which defines IgG Fc chemically; residue 216, taking the first residue of heavy chain constant region to be 114 (Kobet et al., supra), or analogous sites of other immunoglobulins) is used in the fusion. In some embodiments, the SEMA3E sema domain sequence is fused to the hinge region and C_{H}2 and C_{H}3 or C_{H}1, hinge, C_{H}2 and C_{H}3 domains of an IgG1, IgG2, or IgG3 heavy chain. The precise site at which the fusion is made is not critical, and the optimal site can be determined by routine experimentation. In some embodiments, the nucleic acid encodes a linker peptide between the SEMA3E sema domain sequences and the Fc domain.

For human immunoadhesins, the use of human IgG1 and IgG3 immunoglobulin sequences is preferred. A major advantage of using IgG1 is that IgG1 immunoadhesins can be purified efficiently on immobilized protein A. In contrast, purification of IgG3 requires protein G, a significantly less versatile medium. However, other structural and functional properties of immunoglobulins should be considered when choosing the Ig fusion partner for a particular immunoadhesin construction. For example, the IgG3 hinge is longer and more flexible, so it can accommodate larger “adhesin” domains that may not fold or function properly when fused to IgG1. Another consideration may be valency; IgG immunoadhesins are bivalent homodimers, whereas Ig subtypes like IgA and IgM may give rise to dimeric or pentameric structures, respectively, of the basic Ig homodimer unit.

For SEMA3E immunoadhesins designed for in vivo application, the pharmacokinetic properties and the effector functions specified by the Fc region are important as well. Although IgG1, IgG2 and IgG4 all have in vivo half-lives of 21 days, their relative potencies at activating the complement system are different. IgG4 does not activate complement, and IgG2 is significantly weaker at complement activation than IgG1. Moreover, unlike IgG1, IgG2 does not bind to Fc receptors on mononuclear cells or neutrophils. While IgG3 is optimal for complement activation, its in vivo half-life is approximately one third of the other IgG isotypes.

Another important consideration for immunoadhesins designed to be used as human therapeutics is the number of allotypic variants of the particular isotype. In general, IgG isotypes with fewer serologically-defined allotypes are preferred. For example, IgG1 has only four serologically-defined allotypic sites, two of which (G1m and 2) are located in the Fc region; and one of these sites G1m1, is non-immunogenic. In contrast, there are 12 serologically-defined allotypes in IgG3, all of which are in the Fc region; only three of these sites (G3m5, 11 and 21) have one allotype which is nonimmunogenic. Thus, the potential immunogenicity of an IgG3 immunoadhesin is greater than that of an IgG1 immunoadhesin.

The cDNAs encoding the SEMA3E sequence (e.g. a sema domain sequence) and the Ig parts of the immunoadhesin are inserted in tandem into an appropriate vector that directs efficient expression in the chosen host cells.

In some embodiments, a chimeric heteroadhesin polypeptide comprises a fusion of a monomer of the chimeric heteroadhesin with a tag polypeptide which provides an epitope to which an anti-tag antibody can selectively bind. Such epitope tagged forms of the chimeric heteroadhesin are useful, as the presence thereof can be detected using a labeled antibody against the tag polypeptide. Also, provision of the epitope tag enables the chimeric heteroadhesin to be readily purified by affinity purification using the anti-tag antibody. Tag polypeptides and their respective antibodies are well known in the art.

Another type of covalent modification of a chimeric heteromultimer comprises linking a monomer polypeptide of the heteromultimer to one of a variety of non-proteinaceous polymers, e.g., polyethylene glycol, polypropylene glycol, polyoxyalkylenes, or copolymers of polyethylene glycol and polypropylene glycol. A chimeric heteromultimer also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

Selection and Transformation of Host Cells

Host cells are transfected or transformed with expression or cloning vectors described herein for immunoadhesin production and cultured in conventional nutrient media modified as appropriate for inducing promoters, selecting transformants, or amplifying the genes encoding the desired sequences. Suitable host cells for cloning or expressing the DNA in the vectors herein include prokaryotic, fungal, insect, plant, mammalian or other eukaryotic cells. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

The terms “transformation” and “transfection” are used interchangeably herein and refer to the process of introducing DNA into a cell. Following transformation or transfection, the nucleic acid of the invention may integrate into the host cell genome, or may exist as an extrachromosomal element. Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells.

Selection and Use of a Replicable Vector

The nucleic acid encoding immunoadhesin may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The immunoadhesin may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the immunoadhesin-encoding DNA that is inserted into the vector. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. For example, the origin of replication from the plasmid pBR322 is suitable for most Gram-negative bacteria, the 2μ plasmid origin is suitable for yeast, and various viral origins (SV40, polyoma, adenovirus or BPV) are useful for cloning vectors in mammalian cells.

Expression and cloning vectors will typically contain a selection gene, also termed a selectable marker. Typical selection genes encode proteins that (a) confer resistance to antibiotics or other toxins, (b) complement auxotrophic deficiencies, or (c) supply critical nutrients not available from complex media. An example of suitable selectable markers for mammalian cells are those that enable the identification of cells competent to take up the immunoadhesin-encoding nucleic acid, such as DHFR or thymidine kinase. An appropriate host cell when wild-type DHFR is employed is a CHO cell line deficient in DHFR activity.

Expression and cloning vectors usually contain a promoter operably linked to the immunoadhesin-encoding nucleic acid sequence to direct mRNA synthesis. Promoters recognized by a variety of potential host cells are well known. Promoters suitable for use with prokaryotic hosts include the β-lactamase and lactose promoter systems, alkaline phosphatase, a tryptophan (trp) promoter system, and hybrid promoters such as the tac promoter. Promoters for use in bacterial systems also will contain a Shine-Delgarno sequence operably linked to the DNA encoding immunoadhesin.

Examples of suitable promoter sequences for use with yeast hosts include the promoters for 3-phosphoglycerate kinase or other glycolytic enzymes. Other yeast promoters are the inducible promoters for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization.

The transcription of immunoadhesin from vectors in mammalian host cells is controlled, for example, by promoters obtained from the genomes of viruses such as polyoma virus, fowlpox virus, adenovirus (such as Adenovirus 2), bovine papilloma virus, retrovirus (such as avian sarcoma virus), cytomegalovirus, hepatitis-B virus and Simian Virus 40 (SV40); from heterologous mammalian promoters, e.g., the actin promoter or an immunoglobulin promoter, or from heat-shock promoters, provided such promoters are compatible with the host cell systems.

Transcription of a DNA encoding the immunoadhesin by higher eukaryotes may be increased by inserting an enhancer sequence into the vector. Enhancers are cis-acting elements of DNA, usually about from 10 to 300 bp, which act on a promoter to increase its transcription. Many enhancer sequences are now known from mammalian genes (e.g. globin, elastase, albumin, α-fetoprotein and insulin). Typically, however, one will use an enhancer from a eukaryotic cell virus. Examples include the SV40 enhancer on the late side of the replication origin (by 100-270), the cytomegalovirus early promoter enhancer, the polyoma enhancer on the late side of the replication origin, and adenovirus enhancers. The enhancer may be spliced into the vector at a position 5′ or 3′ to the immunoadhesin coding sequence, but is preferably located at a site 5′ from the promoter.

Expression vectors used in eukaryotic host cells will also contain sequences necessary for the termination of transcription and for stabilizing the mRNA. Such sequences are commonly available from the 3′ untranslated regions of eukaryotic or viral DNAs or cDNAs.

Purification and Characterization of Immunoadhesin

An immunoadhesin or a chimeric heteroadhesin preferably is recovered from the culture medium as a secreted polypeptide, although it also may be recovered from host cell lysates. As a first step, the particulate debris, either host cells or lysed fragments, is removed, for example, by centrifugation or ultrafiltration; optionally, the protein may be concentrated with a commercially available protein concentration filter, followed by separating the chimeric heteroadhesin from other impurities by one or more purification procedures selected from: fractionation on an immunoaffinity column; fractionation on an ion-exchange column; ammonium sulphate or ethanol precipitation; reverse phase HPLC; chromatography on silica; chromatography on heparin Sepharose™; chromatography on a cation exchange resin; chromatofocusing; SDS-PAGE; and gel filtration.

A particularly advantageous method of purifying immunoadhesins is affinity chromatography. The choice of affinity ligand depends on the species and isotype of the immunoglobulin Fc domain that is used in the chimera. Protein A can be used to purify immunoadhesins that are based on human IgG1, IgG2, or IgG4 heavy chains (Lindmark et al., 1983, J. Immunol. Meth. 62, 1-13). Protein G is recommended for all mouse isotypes and for human IgG3 (Guss et al., 1986, EMBO J. 5, 1567-75). The matrix to which the affinity ligand is attached is most often agarose, but other matrices are also available. Mechanically stable matrices such as controlled pore glass or poly(styrenedivinyl)benzene allow for faster flow rates and shorter processing times than can be achieved with agarose. The conditions for binding an immunoadhesin to the protein A or G affinity column are dictated entirely by the characteristics of the Fc domain; that is, its species and isotype. Generally, when the proper ligand is chosen, efficient binding occurs directly from unconditioned culture fluid. One distinguishing feature of immunoadhesins is that, for human IgG1 molecules, the binding capacity for protein A is somewhat diminished relative to an antibody of the same Fc type. Bound immunoadhesin can be efficiently eluted either at acidic pH (at or above 3.0), or in a neutral pH buffer containing a mildly chaotropic salt. This affinity chromatography step can result in an immunoadhesin preparation that is >95% pure.

Other methods known in the art can be used in place of, or in addition to, affinity chromatography on protein A or G to purify immunoadhesins. Immunoadhesins behave similarly to antibodies in thiophilic gel chromatography (Hutchens and Porath, 1986, Anal. Biochem. 159: 217-26) and immobilized metal chelate chromatography (Al-Mashikhi and Makai, 1988, J. Dairy Sci. 71: 1756-63). In contrast to antibodies, however, their behavior on ion exchange columns is dictated not only by their isoelectric points, but also by a charge dipole that may exist in the molecules due to their chimeric nature.

In some embodiments, the SEMA3E immunoadhesins are assembled as monomers, or hetero- or homo-multimers, dimers or tetramers, essentially as illustrated in WO 91/08298. Generally, these assembled immunoglobulins will have known unit structures. A basic four chain structural unit is the form in which IgG, IgD, and IgE exist. A four-unit structure is repeated in the higher molecular weight immunoglobulins; IgM generally exists as a pentamer of basic four units held together by disulfide bonds. IgA globulin, and occasionally IgG globulin, may also exist in multimeric form in serum. In the case of multimer, each four unit may be the same or different.

The SEMA3E immunoadhesins of the invention will agonize PLEXIND1 activity.

Antibodies

In some embodiments, the anti-PLEXIND1 agonist is an antibody, typically a monoclonal antibody. Also encompassed within the scope of the invention are Fab, Fab′, Fab′-SH and F(ab′)2 fragments of such anti-PLEXIND1 agonist antibodies. These antibody fragments can be created by traditional means, such as enzymatic digestion, or may be generated by recombinant techniques. Such antibody fragments may be chimeric or humanized. These fragments are useful for the purposes set forth herein.

Anti-PLEXIND1 agonist monoclonal antibodies can be made using the hybridoma method first described by Kohler et al., 1975, Nature, 256: 495, or may be made by recombinant DNA methods (e.g., U.S. Pat. No. 4,816,567).

In the hybridoma method, a mouse or other appropriate host animal, such as a hamster, is immunized to elicit lymphocytes that produce or are capable of producing antibodies that will specifically bind to the protein used for immunization. Antibodies to PLEXIND1 generally are raised in animals by multiple subcutaneous (sc) or intraperitoneal (ip) injections of PLEXIND1 and an adjuvant. PLEXIND1 may be prepared using methods well-known in the art, some of which are further described herein. In some embodiments, animals are immunized with a derivative of PLEXIND1 that contains the extracellular domain (ECD) of PLEXIND1 fused to the Fc portion of an immunoglobulin heavy chain. In another embodiment, animals are immunized with a PLEXIND1-IgG1 fusion protein. Animals ordinarily are immunized against immunogenic conjugates or derivatives of PLEXIND1 with monophosphoryl lipid A (MPL)/trehalose dicorynomycolate (TDM) (Ribi Immunochem. Research, Inc., Hamilton, Mont.) and the solution is injected intradermally at multiple sites. Two weeks later the animals are boosted. 7 to 14 days later animals are bled and the serum is assayed for anti-PLEXIND1 titer. Animals are boosted until titer plateaus.

Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, 1984, J. Immunol., 133: 3001; Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against PLEXIND1. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by the Scatchard analysis. The agonist activity of the monoclonal antibodies may be determined by measuring their ability to inhibit PLEXIND1-mediated endothelial cell migration, e.g. using an assay such as described in Examples 3 or 5.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose™, hydroxylapatite chromatography, gel electrophoresis, dialysis, or affinity chromatography.

Anti-PLEXIND1 agonist antibodies can also be made by using combinatorial libraries to screen for synthetic antibody clones with the desired activity or activities. In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the anti-PLEXIND1 antibodies can be obtained by designing a suitable antigen screening procedure to select for the phage clone of interest followed by construction of a full length anti-PLEXIND1 antibody clone using the Fv sequences from the phage clone of interest and suitable constant region (Fc) sequences described in Kabat et al., Sequences of Proteins of Immunological Interest, Fifth Edition, NIH Publication 91-3242, Bethesda Md. (1991), vols. 1-3.

The antigen-binding domain of an antibody is formed from two variable (V) regions of about 110 amino acids, one each from the light (V_{L}) and heavy (V_{H}) chains, that both present three hypervariable loops or complementarity-determining regions (CDRs). Variable domains can be displayed functionally on phage, either as single-chain Fv (scFv) fragments, in which V_{H} and V_{L} are covalently linked through a short, flexible peptide, or as Fab fragments, in which they are each fused to a constant domain and interact non-covalently, as described in Winter et al., 1994, Ann. Rev. Immunol., 12: 433-55. As used herein, scFv encoding phage clones and Fab encoding phage clones are collectively referred to as “Fv phage clones” or “Fv clones”.

Repertoires of V_{H} and V_{L} genes can be separately cloned by polymerase chain reaction (PCR) and recombined randomly in phage libraries, which can then be searched for antigen-binding clones as described in Winter et al., 1994, Ann. Rev. Immunol., 12: 433-55. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., 1993, EMBO J., 12: 725-34. Finally, naive libraries can also be made synthetically by cloning the unrearranged V-gene segments from stem cells, and using PCR primers containing random sequence to encode the highly variable CDR3 regions and to accomplish rearrangement in vitro as described by Hoogenboom and Winter, 1992, J. Mol. Biol., 227: 381-88.

Filamentous phage is used to display antibody fragments by fusion to the minor coat protein pIII. The antibody fragments can be displayed as single chain Fv fragments, in which VH and VL domains are connected on the same polypeptide chain by a flexible polypeptide spacer, e.g. as described by Marks et al., 1991, J. Mol. Biol., 222: 581-97, or as Fab fragments, in which one chain is fused to pIII and the other is secreted into the bacterial host cell periplasm where assembly of a Fab-coat protein structure which becomes displayed on the phage surface by displacing some of the wild type coat proteins, e.g. as described in Hoogenboom et al., Nucl. Acids Res., 1991, 19: 4133-37.

In general, nucleic acids encoding antibody gene fragments are obtained from immune cells harvested from humans or animals. If a library biased in favor of anti-PLEXIND1 clones is desired, the subject is immunized with PLEXIND1 to generate an antibody response, and spleen cells and/or circulating B cells other peripheral blood lymphocytes (PBLs) are recovered for library construction. In a preferred embodiment, a human antibody gene fragment library biased in favor of anti-PLEXIND1 clones is obtained by generating an anti-PLEXIND1 antibody response in transgenic mice carrying a functional human immunoglobulin gene array (and lacking a functional endogenous antibody production system) such that PLEXIND1 immunization gives rise to B cells producing human antibodies against PLEXIND1. The generation of human antibody-producing transgenic mice is described below.

Additional enrichment for anti-PLEXIND1 reactive cell populations can be obtained by using a suitable screening procedure to isolate B cells expressing PLEXIND1-specific membrane bound antibody, e.g., by cell separation with PLEXIND1 affinity chromatography or adsorption of cells to fluorochrome-labeled PLEXIND1 followed by flow-activated cell sorting (FACS).

Alternatively, the use of spleen cells and/or B cells or other PBLs from an unimmunized donor provides a better representation of the possible antibody repertoire, and also permits the construction of an antibody library using any animal (human or non-human) species in which PLEXIND1 is not antigenic. For libraries incorporating in vitro antibody gene construction, stem cells are harvested from the subject to provide nucleic acids encoding unrearranged antibody gene segments. The immune cells of interest can be obtained from a variety of animal species, such as human, mouse, rat, lagomorpha, lupine, canine, feline, porcine, bovine, equine, and avian species, etc.

Nucleic acid encoding antibody variable gene segments (including V_{H} and V_{L} segments) are recovered from the cells of interest and amplified. In the case of rearranged V_{H} and V_{L} gene libraries, the desired DNA can be obtained by isolating genomic DNA or mRNA from lymphocytes followed by polymerase chain reaction (PCR) with primers matching the 5′ and 3′ ends of rearranged V_{H} and V_{L} genes as described in Orlandi et al., 1989, Proc. Natl. Acad. Sci. USA, 86: 3833-37, thereby making diverse V gene repertoires for expression. The V genes can be amplified from cDNA and genomic DNA, with back primers at the 5′ end of the exon encoding the mature V-domain and forward primers based within the J-segment as described in Orlandi et al. (1989) and in Ward et al., 1989, Nature, 341: 544-46. However, for amplifying from cDNA, back primers can also be based in the leader exon as described in Jones et al., 1991, Biotechnol., 9: 88-89, and forward primers within the constant region as described in Sastry et al., 1989, Proc. Natl. Acad. Sci. USA, 86: 5728-32. To maximize complementarity, degeneracy can be incorporated in the primers as described in Orlandi et al. (1989) or Sastry et al. (1989). Preferably, the library diversity is maximized by using PCR primers targeted to each V-gene family in order to amplify all available V_{H} and V_{L} arrangements present in the immune cell nucleic acid sample, e.g. as described in the method of Marks et al., 1991, J. Mol. Biol., 222: 581-97 or as described in the method of Orum et al., 1993, Nucleic Acids Res., 21: 4491-98. For cloning of the amplified DNA into expression vectors, rare restriction sites can be introduced within the PCR primer as a tag at one end as described in Orlandi et al. (1989), or by further PCR amplification with a tagged primer as described in Clackson et al., 1991, Nature, 352: 624-28.

Repertoires of synthetically rearranged V genes can be derived in vitro from V gene segments. Most of the human V_{H}-gene segments have been cloned and sequenced (reported in Tomlinson et al., 1992, J. Mol. Biol., 227: 776-98), and mapped (reported in Matsuda et al., 1993, Nature Genet., 3: 88-94; these cloned segments (including all the major conformations of the H1 and H2 loop) can be used to generate diverse V_{H} gene repertoires with PCR primers encoding H3 loops of diverse sequence and length as described in Hoogenboom and Winter, 1992, J. Mol. Biol., 227: 381-88. V_{H} repertoires can also be made with all the sequence diversity focused in a long H3 loop of a single length as described in Barbas et al., 1991, Proc. Natl. Acad. Sci. USA, 89: 4457-61. Human Vκ and Vλ segments have been cloned and sequenced (reported in Williams and Winter, 1993, Eur. J. Immunol., 23: 1456-61) and can be used to make synthetic light chain repertoires. Synthetic V gene repertoires, based on a range of V_{H} and V_{L} folds, and L3 and H3 lengths, will encode antibodies of considerable structural diversity. Following amplification of V-gene encoding DNAs, germline V-gene segments can be rearranged in vitro according to the methods of Hoogenboom and Winter, 1992, J. Mol. Biol., 227: 381-88.

Repertoires of antibody fragments can be constructed by combining V_{H} and V_{L} gene repertoires together in several ways. Each repertoire can be created in different vectors, and the vectors recombined in vitro, e.g., as described in Hogrefe et al., 1993, Gene, 128: 119-26, or in vivo by combinatorial infection, e.g., the loxP system described in Waterhouse et al., 1993, Nucl. Acids Res., 21: 2265-66. The in vivo recombination approach exploits the two-chain nature of Fab fragments to overcome the limit on library size imposed by E. coli transformation efficiency. Naive V_{H} and V_{L} repertoires are cloned separately, one into a phagemid and the other into a phage vector. The two libraries are then combined by phage infection of phagemid-containing bacteria so that each cell contains a different combination and the library size is limited only by the number of cells present (about 10¹² clones). Both vectors contain in vivo recombination signals so that the V_{H} and V_{L} genes are recombined onto a single replicon and are co-packaged into phage virions. These huge libraries provide large numbers of diverse antibodies of good affinity (K_{d} of about 10⁻⁸ M).

Alternatively, the repertoires may be cloned sequentially into the same vector, e.g. as described in Barbas et al., 1991, Proc. Natl. Acad. Sci. USA, 88: 7978-82, or assembled together by PCR and then cloned, e.g. as described in Clackson et al., 1991, Nature, 352: 624-28. PCR assembly can also be used to join V_{H} and V_{L} DNAs with DNA encoding a flexible peptide spacer to form single chain Fv (scFv) repertoires. In yet another technique, “in cell PCR assembly” is used to combine V_{H} and V_{L} genes within lymphocytes by PCR and then clone repertoires of linked genes as described in Embleton et al., 1992, Nucl. Acids Res., 20: 3831-37.

The antibodies produced by naive libraries (either natural or synthetic) can be of moderate affinity (K_{d} of about 10⁻⁶ to 10⁻⁷ M), but affinity maturation can also be mimicked in vitro by constructing and reselecting from secondary libraries as described in Winter et al. (1994), supra. For example, mutation can be introduced at random in vitro by using error-prone polymerase (Leung et al., 1989, Technique, 1: 11-15 (1989)); Hawkins et al., 1992, J. Mol. Biol., 226: 889-896 (1992); Gram et al., 1992, Proc. Natl. Acad. Sci. USA, 89: 3576-80. Additionally, affinity maturation can be performed by randomly mutating one or more CDRs, e.g. using PCR with primers carrying random sequence spanning the CDR of interest, in selected individual Fv clones and screening for higher affinity clones. WO 96/07754 described a method for inducing mutagenesis in a complementarity determining region of an immunoglobulin light chain to create a library of light chain genes. Another effective approach is to recombine the V_{H} or V_{L} domains selected by phage display with repertoires of naturally occurring V domain variants obtained from unimmunized donors and screen for higher affinity in several rounds of chain reshuffling as described in Marks et al., 1992, Biotechnol., 10: 779-83. This technique allows the production of antibodies and antibody fragments with affinities in the 10⁻⁹ M range.

PLEXIND1 nucleic acid and amino acid sequences are known in the art. GenBank® accession numbers for representative PLEXIND1 polypeptide and nucleic acid sequences are: AY116661, BC062465, BCO11848 (human), BC019530, AL603923, and AY688678 (mouse). DNAs encoding PLEXIND1 can be prepared by a variety of methods known in the art. These methods include, but are not limited to, chemical synthesis or isolation from a genomic or cDNA library.

Following construction of the DNA molecule encoding the PLEXIND1, the DNA molecule is used to generate protein according to known methods and as generally described hereinbefore.

The phage library samples are contacted with immobilized PLEXIND1 under conditions suitable for binding of at least a portion of the phage particles with the adsorbent. Normally, the conditions, including pH, ionic strength, temperature and the like are selected to mimic physiological conditions. The phages bound to the solid phase are washed and then eluted by acid, e.g. as described in Barbas et al., 1991, Proc. Natl. Acad. Sci. USA, 88: 7978-82, or by alkali, e.g. as described in Marks et al., 1991, J. Mol. Biol., 222: 581-97, or by PLEXIND1 antigen competition, e.g. in a procedure similar to the antigen competition method of Clackson et al., 1991, Nature, 352: 624-28. Phages can be enriched 20-1,000-fold in a single round of selection. Moreover, the enriched phages can be grown in bacterial culture and subjected to further rounds of selection.

The efficiency of selection depends on many factors, including the kinetics of dissociation during washing, and whether multiple antibody fragments on a single phage can simultaneously engage with antigen. Antibodies with fast dissociation kinetics (and weak binding affinities) can be retained by use of short washes, multivalent phage display and high coating density of antigen in solid phase. The high density not only stabilizes the phage through multivalent interactions, but favors rebinding of phage that has dissociated. The selection of antibodies with slow dissociation kinetics (and good binding affinities) can be promoted by use of long washes and monovalent phage display as described in Bass et al., 1990, Proteins, 8: 309-14 and in WO 92/09690, and a low coating density of antigen as described in Marks et al., 1992, Biotechnol., 10: 779-83.

It is possible to select between phage antibodies of different affinities, even with affinities that differ slightly, for PLEXIND1. However, random mutation of a selected antibody (e.g. as performed in some of the affinity maturation techniques described above) is likely to give rise to many mutants, most binding to antigen, and a few with higher affinity. With limiting PLEXIND1, rare high affinity phage could be competed out. To retain all the higher affinity mutants, phages can be incubated with excess biotinylated PLEXIND1, but with the biotinylated PLEXIND1 at a concentration of lower molarity than the target molar affinity constant for PLEXIND1. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity. Conditions used in washing phages bound to a solid phase can also be manipulated to discriminate on the basis of dissociation kinetics.

DNA encoding the hybridoma-derived monoclonal antibodies or phage display Fv clones is readily isolated and sequenced using conventional procedures (e.g. by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells. Review articles on recombinant expression in bacteria of antibody-encoding DNA include Skerra et al., 1993, Curr. Opinion in Immunol., 5: 256 and Plückthun, 1992, Immunol. Rev., 130: 151.

DNA encoding the Fv clones can be combined with known DNA sequences encoding heavy chain and/or light chain constant regions (e.g. the appropriate DNA sequences can be obtained from Kabat et al., supra) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. A Fv clone derived from the variable domain DNA of one animal (such as human) species and then fused to constant region DNA of another animal species to form coding sequence(s) for “hybrid”, full length heavy chain and/or light chain is included in the definition of “chimeric” and “hybrid” antibody as used herein. In a preferred embodiment, a Fv clone derived from human variable DNA is fused to human constant region DNA to form coding sequence(s) for all human, full or partial length heavy and/or light chains.

DNA encoding anti-PLEXIND1 antibody derived from a hybridoma can also be modified, for example, by substituting the coding sequence for human heavy- and light-chain constant domains in place of homologous murine sequences derived from the hybridoma clone (e.g. Morrison et al., 1984, Proc. Natl. Acad. Sci. USA, 81: 6851-55). DNA encoding a hybridoma or Fv clone-derived antibody or fragment can be further modified by covalently joining to the immunoglobulin coding sequence all or part of the coding sequence for a non-immunoglobulin polypeptide. In this manner, “chimeric” or “hybrid” antibodies are prepared that have the binding specificity of the Fv clone or hybridoma clone-derived antibodies.

Antibody Fragments

The present invention encompasses antibody fragments. In certain circumstances there are advantages of using antibody fragments, rather than whole antibodies. The smaller size of the fragments allows for rapid clearance, and may lead to improved access to target sites.

Various techniques have been developed for the production of antibody fragments. Traditionally, these fragments were derived via proteolytic digestion of intact antibodies (see, e.g., Morimoto et al., 1992, J. Biochem. Biophys. Meth. 24:107-17; and Brennan et al., 1985, Science, 229:81). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., 1992, Bio/Technology 10: 163-67). According to another approach, F(ab′)2 fragments can be isolated directly from recombinant host cell culture. Fab and F(ab′)2 fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

Humanized Antibodies

The present invention encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., 1986, Nature 321:522-25; Riechmann et al., 1988, Nature 332:323-27; Verhoeyen et al., 1988, Science 239:1534-36), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody (Sims et al., 1993, J. Immunol. 151:2296; Chothia et al., 1987, J. Mol. Biol. 196:901. Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA, 89:4285; Presta et al., 1993, J. Immunol., 151:2623.

It is further important that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human Antibodies

Human anti-PLEXIND1 antibodies can be constructed by combining Fv clone variable domain sequence(s) selected from human-derived phage display libraries with known human constant domain sequences(s) as described above. Alternatively, human monoclonal anti-PLEXIND1 antibodies can be made by the hybridoma method.

It is now possible to produce transgenic animals (e.g. mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., 1993, Proc. Natl. Acad. Sci. USA 90: 2551; Jakobovits et al., 1993, Nature 362: 255; Bruggermann et al., 1993, Year in Immunol. 7: 33.

Gene shuffling can also be used to derive human antibodies from non-human, e.g. rodent, antibodies, where the human antibody has similar affinities and specificities to the starting non-human antibody. According to this method, which is also called “epitope imprinting”, either the heavy or light chain variable region of a non-human antibody fragment obtained by phage display techniques as described above is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e. the epitope governs (imprints) the choice of the human chain partner. When the process is repeated in order to replace the remaining non-human chain, a human antibody is obtained (see WO 93/06213). Unlike traditional humanization of non-human antibodies by CDR grafting, this technique provides completely human antibodies, which have no FR or CDR residues of non-human origin.

Antibody Variants

In some embodiments, amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid alterations may be introduced in the subject antibody amino acid sequence at the time that sequence is made.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US 2003/0157108. See also US 2004/0093621. Antibodies with a bisecting N-acetylglucosamine (GlcNAc) in the carbohydrate attached to an Fc region of the antibody are referenced in WO 2003/011878 and U.S. Pat. No. 6,602,684. Antibodies with at least one galactose residue in the oligosaccharide attached to an Fc region of the antibody are reported in WO 97/30087. See, also, WO 98/58964 and WO 99/22764 concerning antibodies with altered carbohydrate attached to the Fc region thereof. See also US 2005/0123546 on antigen-binding molecules with modified glycosylation.

The preferred glycosylation variant herein comprises an Fc region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al., 2004, J. Mol. Biol. 336: 1239-49 (2004); Yamane-Ohnuki et al., 2004, Biotech. Bioeng. 87: 614. Examples of cell lines producing defucosylated antibodies include Lec13 CHO cells deficient in protein fucosylation (Ripka et al., 1986, Arch. Biochem. Biophys. 249: 533-45; US 2003/0157108 A1; and WO 2004/056312 A1 (especially at Example 11), and knockout cell lines, such as alpha-1,6-fucosyltransferase gene (FUT8) knockout CHO cells (Yamane-Ohnuki et al., 2004, Biotech. Bioeng. 87: 614).

It may be desirable to introduce one or more amino acid modifications in an Fc region of the immunoglobulin polypeptides, thereby generating a Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

In accordance with this description and the teachings of the art, it is contemplated that in some embodiments, an antibody used in methods may comprise one or more alterations as compared to the wild-type counterpart antibody, e.g. in the Fc region. These antibodies would nonetheless retain substantially the same characteristics required for therapeutic utility as compared to their wild-type counterpart. For example, it is thought that certain alterations can be made in the Fc region that would result in altered (i.e., either improved or diminished) C1q binding and/or Complement Dependent Cytotoxicity (CDC), e.g., as described in WO99/51642. See also Duncan & Winter, 1988, Nature 322: 738-40; U.S. Pat. No. 5,648,260; U.S. Pat. No. 5,624,821; and WO94/29351 concerning other examples of Fc region variants. WO00/42072 and WO 2004/056312 describe antibody variants with improved or diminished binding to FcRs. See, also, Shields et al., 2001, J. Biol. Chem. 9(2): 6591-604. Antibodies with increased half lives and improved binding to the neonatal Fc receptor (FcRn), which is responsible for the transfer of maternal IgGs to the fetus (Guyer et al., 1976, J. Immunol. 117: 587 and Kim et al., 1994, J. Immunol. 24: 249), are described in US2005/0014934A1 (Hinton et al.). These antibodies comprise an Fc reg on with one or more substitutions therein which improve binding of the Fc region to FcRn. Polypeptide variants with altered Fc region amino acid sequences and increased or decreased C1q binding capability are described in U.S. Pat. No. 6,194,551, WO99/51642. See, also, Idusogie et al., 2000, J. Immunol. 164: 4178-84.

Antibody Derivatives

The antibodies can be further modified to contain additional nonproteinaceous moieties that are known in the art and readily available. Preferably, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymers are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

Screening for Antibodies with Desired Properties

The antibodies can be characterized for their physical/chemical properties and biological functions by various assays known in the art. In some embodiments, antibodies are characterized for any one or more of: binding to PLEXIND1, inhibition of angiogenesis, treatment and/or prevention of a tumor, cell proliferative disorder or a cancer. For use in the methods of the invention, the anti-PLEXIND1 antibodies are PLEXIND1 agonists.

The purified antibodies can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

In certain embodiments of the invention, the antibodies produced herein are analyzed for their biological activity. In some embodiments, the antibodies of the present invention are tested for their antigen binding activity. The antigen binding assays that are known in the art and can be used herein include without limitation any direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays.

Vectors, Host Cells and Recombinant Methods

For recombinant production of an antibody, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin. It will be appreciated that constant regions of any isotype can be used for this purpose, including IgG, IgM, IgA, IgD, and IgE constant regions, and that such constant regions can be obtained from any human or animal species.

Generating Antibodies Using Prokaryotic Host Cells:

Vector Construction

Polynucleotide sequences encoding polypeptide components of the antibody can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of antibodies are described in U.S. Pat. No. 5,648,237.

In addition, phage vectors containing replicon and control sequences that are compatible with the host microorganism can be used as transforming vectors in connection with these hosts. For example, bacteriophage such as λGEM™-1 may be utilized in making a recombinant vector which can be used to transform susceptible host cells such as E. coli LE392.

The expression vector may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the β-galactanase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al., 1980, Cell 20: 269) using linkers or adaptors to supply any required restriction sites.

Each cistron within the recombinant vector may comprise a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In some embodiments of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB strains) provide cytoplasm conditions that are favorable for disulfide bond formation, thereby permitting proper folding and assembly of expressed protein subunits. Proba & Plückthun, 1995, Gene 159: 203.

Prokaryotic host cells suitable for expressing antibodies include, e.g., Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. It is generally necessary to select the appropriate bacteria taking into consideration replicability of the replicon in the cells of a bacterium. Typically the host cell should secrete minimal amounts of proteolytic enzymes, and additional protease inhibitors may desirably be incorporated in the cell culture.

Antibody Production

Host cells are transformed with the above-described expression vectors and cultured in conventional nutrient media modified as appropriate for inducing promoters, selecting transformants, or amplifying the genes encoding the desired sequences.

Prokaryotic cells used to produce the polypeptides are grown in media known in the art and suitable for culture of the selected host cells. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector, protein expression is induced under conditions suitable for the activation of the promoter, e.g. PhoA promoters are used for controlling transcription of the polypeptides by culturing under phosphate-limiting conditions. A variety of other inducers may be used, according to the vector construct employed, as is known in the art.

In some embodiments, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the invention, antibody production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and/or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al., 1999, J. Biol. Chem. 274:19601-19605; U.S. Pat. No. 6,083,715; U.S. Pat. No. 6,027,888; Bothmann and Plückthun, 2000, J. Biol. Chem. 275:17100-17105; Ramm and Plückthun, 2000, J. Biol. Chem. 275:17106-17113; Arie et al., 2001. Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; U.S. Pat. No. 5,264,365; Georgiou et al., U.S. Pat. No. 5,508,192; Hara et al., 1996, Microbial Drug Resistance, 2:63-72.

In some embodiments, E. coli strains deficient for proteolytic enzymes and transformed with plasmids overexpressing one or more chaperone proteins are used as host cells in the expression system.

Antibody Purification

Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one aspect, Protein A immobilized on a solid phase is used for immunoaffinity purification of the full length antibody products. Protein A is a 41kD cell wall protein from Staphylococcus aureas which binds with a high affinity to the Fc region of antibodies. Lindmark et al., 1983, J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants.

As the first step of purification, the preparation derived from the cell culture as described above is applied onto the Protein A immobilized solid phase to allow specific binding of the antibody of interest to Protein A. The solid phase is then washed to remove contaminants non-specifically bound to the solid phase. Finally the antibody of interest is recovered from the solid phase by elution.

Generating Antibodies Using Eukaryotic Host Cells

The vector components generally include, but are not limited to, one or more of the following: a signal sequence, an origin of replication, one or more marker genes, an enhancer element, a promoter, and a transcription termination sequence.

Signal Sequence Component

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

Origin of Replication

Generally, an origin of replication component is not needed for mammalian expression vectors. For example, the SV40 origin may typically be used only because it contains the early promoter.

Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

An example of suitable selectable markers for mammalian cells are those that enable the identification of cells competent to take up the antibody nucleic acid, such as DHFR, thymidine kinase, metallothionein-I and -II, preferably primate metallothionein genes, adenosine deaminase, ornithine decarboxylase, etc. For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Promoter Component

Expression and cloning vectors usually contain a promoter that is recognized by the host organism and is operably linked to the antibody polypeptide nucleic acid. Promoter sequences are known for eukaryotes.

Antibody polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

Enhancer Element Component

Transcription of DNA encoding the antibody polypeptide of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, 1982, Nature 297: 17-18 on enhancing elements for activation of eukaryotic promoters. The enhancer may be spliced into the vector at a position 5′ or 3′ to the antibody polypeptide-encoding sequence, but is preferably located at a site 5′ from the promoter.

Transcription Termination Component

Expression vectors used in eukaryotic host cells will typically also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure.

Host cells are transformed with the above-described expression or cloning vectors for antibody production and cultured in conventional nutrient media modified as appropriate for inducing promoters, selecting transformants, or amplifying the genes encoding the desired sequences.

Culturing the Host Cells

The host cells used to produce an antibody of this invention may be cultured in a variety of media. For example, commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Purification of Antibody

When using recombinant techniques, the antibody can be produced intracellularly, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., 1983, J. Immunol. Meth. 62: 1-13). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

Immunoconjugates

The invention contemplates immunoconjugates (interchangeably termed “antibody-drug conjugates” or “ADC”), comprising an anti-PLEXIND1 antibody conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

The use of antibody-drug conjugates for the local delivery of cytotoxic or cytostatic agents, i.e. drugs to kill or inhibit tumor cells in the treatment of cancer (Syrigos & Epenetos, 1999, Anticancer Res. 19:605-14; Niculescu-Duvaz & Springer, 1997, Adv. Drug Del. Rev. 26:151-72; U.S. Pat. No. 4,975,278) allows targeted delivery of the drug moiety to tumors, and intracellular accumulation therein, where systemic administration of these unconjugated drug agents may result in unacceptable levels of toxicity to normal cells as well as the tumor cells sought to be eliminated. Maximal efficacy with minimal toxicity is sought thereby. Both polyclonal antibodies and monoclonal antibodies have been reported as useful in these strategies (Rowland et al., 1986, Cancer Immunol. Immunother., 21:183-87). Drugs used in these methods include daunomycin, doxorubicin, methotrexate, and vindesine (Rowland et al., 1986, supra). Toxins used in antibody-toxin conjugates include bacterial toxins such as diphtheria toxin, plant toxins such as ricin, small molecule toxins such as geldanamycin (Mandler et al., 2000, J. Nat. Cancer Inst. 92(19): 1573-81; Mandler et al., 2000, Bioorganic & Med. Chem. Lett, 10: 1025-28; Mandler et al., 2002, Bioconjugate Chem. 13: 786-91), maytansinoids (EP 1391213; Liu et al., 1996, Proc. Natl. Acad. Sci. USA 93: 8618-23), and calicheamicin (Lode et al., 1998, Cancer Res. 58: 2928; Hinman et al., 1993, Cancer Res. 53: 3336-42). The toxins may affect their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Preparation of Antibody Drug Conjugates

In the antibody drug conjugates (ADC), an antibody (Ab) is conjugated to one or more drug moieties (D), e.g. about 1 to about 20 drug moieties per antibody, through a linker (L). The ADC of Formula I may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Additional methods for preparing ADC are described herein.

Ab-(L-D)ₚ I

The linker may be composed of one or more linker components. Exemplary linker components include 6-maleimidocaproyl (“MC”), maleimidopropanoyl (“MP”), valine-citrulline (“val-cit”), alanine-phenylalanine (“ala-phe”), p-aminobenzyloxycarbonyl (“PAB”), N-Succinimidyl 4-(2-pyridylthio)pentanoate (“SPP”), N-Succinimidyl 4-(N-maleimidomethyl)cyclohexane-1 carboxylate (“SMCC”), and N-Succinimidyl (4-iodo-acetyl)aminobenzoate (“SIAB”). Additional linker components are known in the art and some are described herein. See also US 2005/0238649.

In some embodiments, the linker may comprise amino acid residues. Exemplary amino acid linker components include a dipeptide, a tripeptide, a tetrapeptide or a pentapeptide. Exemplary dipeptides include: valine-citrulline (vc or val-cit), alanine-phenylalanine (af or ala-phe). Exemplary tripeptides include: glycine-valine-citrulline (gly-val-cit) and glycine-glycine-glycine (gly-gly-gly). Amino acid residues which comprise an amino acid linker component include those occurring naturally, as well as minor amino acids and non-naturally occurring amino acid analogs, such as citrulline. Amino acid linker components can be designed and optimized in their selectivity for enzymatic cleavage by a particular enzymes, for example, a tumor-associated protease, cathepsin B, C and D, or a plasmin protease.

Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues).

Antibody drug conjugates may also be produced by modification of the antibody to introduce electrophilic moieties, which can react with nucleophilic substituents on the linker reagent or drug. The sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In some embodiments, reaction of the carbohydrate portion of a glycosylated antibody with either galactose oxidase or sodium meta-periodate may yield carbonyl (aldehyde and ketone) groups in the protein that can react with appropriate groups on the drug (Hermanson, Bioconjugate Techniques). In another embodiment, proteins containing N-terminal serine or threonine residues can react with sodium meta-periodate, resulting in production of an aldehyde in place of the first amino acid (Geoghegan & Stroh, 1992, Bioconjugate Chem. 3: 138-46; U.S. Pat. No. 5,362,852). Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and acylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In yet another embodiment, the antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Covalent Modifications to SEMA3E Polypeptides

Covalent modifications of the polypeptide agonists of the invention (e.g., a polypeptide agonist fragment, a chimeric SEMA3E molecule (e.g., an SEMA3E immunoadhesin), an anti-PLEXIND1 antibody), are included within the scope of this invention. They may be made by chemical synthesis or by enzymatic or chemical cleavage of the polypeptide, if applicable. Other types of covalent modifications of the polypeptide are introduced into the molecule by reacting targeted amino acid residues of the polypeptide with an organic derivatizing agent that is capable of reacting with selected side chains or the N- or C-terminal residues, or by incorporating a modified amino acid or unnatural amino acid into the growing polypeptide chain, e.g., Ellman et al., 1991, Meth. Enzym. 202: 301-36; Noren et al., 1989, Science 244: 182; and US 2003/0108885 and US 2003/0082575.

Cystinyl residues most commonly are reacted with α-haloacetates (and corresponding amines), such as chloroacetic acid or chloroacetamide, to give carboxymethyl or carboxyamidomethyl derivatives. Cystinyl residues also are derivatized by reaction with bromotrifluoroacetone, α-bromo-β-(5-imidazoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethyl-pyro-carbonate at pH 5.5-7.0 because this agent is relatively specific for the histidyl side chain. Para-bromophenacyl bromide also is useful; the reaction is typically performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and 3-nitro derivatives, respectively. Tyrosyl residues are iodinated using 125I or 131I to prepare labeled proteins for use in radioimmunoassay.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl)carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification involves chemically or enzymatically coupling glycosides to a polypeptide of the invention. These procedures are advantageous in that they do not require production of the polypeptide in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 and in Aplin & Wriston, CRC Crit. Rev. Biochem., pp. 259-306 (1981).

Removal of any carbohydrate moieties present on a polypeptide of the invention may be accomplished chemically or enzymatically. Chemical deglycosylation requires exposure of the polypeptide to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin, et al., 1987, Arch. Biochem. Biophys. 259: 52 and by Edge et al., 1981, Anal. Biochem., 118: 131. Enzymatic cleavage of carbohydrate moieties, e.g., on antibodies, can be achieved by the use of a variety of endo- and exo-glycosidases as described by Thotakura et al., 1987, Meth. Enzymol. 138: 350.

Another type of covalent modification of a polypeptide of the invention comprises linking the polypeptide to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

Pharmaceutical Formulations

Therapeutic formulations comprising an antibody or immunoadhesin of the invention are prepared for storage by mixing the antibody having the desired degree of purity with optional physiologically acceptable carriers, excipients or stabilizers (Remington: The Science and Practice of Pharmacy 20th edition (2000)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy 20th edition (2000).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the immunoglobulin, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated immunoglobulins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

It is further contemplated that an agent useful in the invention can be introduced to a subject by gene therapy. Gene therapy refers to therapy performed by the administration of a nucleic acid to a subject. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs or siRNA can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., 1986, Proc. Natl. Acad. Sci. USA 83: 4143-46). The oligonucleotides can be modified to enhance their uptake, e.g. by substituting their negatively charged phosphodiester groups by uncharged groups.

Dosage and Administration

The molecules are administered to a subject, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes, and/or subcutaneous administration.

In certain embodiments, the treatment of the invention involves the combined administration of a PLEXIND1 agonist and one or more other agents, e.g., one or more anti-angiogenesis agents, one or more chemotherapeutic agents, etc. In some embodiments, a cocktail of different chemotherapeutic agents is administered with the PLEXIND1 agonist and/or one or more anti-angiogenesis agents. The combined administration includes coadministration, using separate formulations or a single pharmaceutical formulation, and/or consecutive administration in either order. For example, a PLEXIND1 agonist may precede, follow, alternate with administration of the anti-cancer agents, or may be given simultaneously therewith. In some embodiments, there is a time period while both (or all) active agents simultaneously exert their biological activities.

For the prevention or treatment of disease, the appropriate dosage of PLEXIND1 agonist will depend on the type of disease to be treated, as defined above, the severity and course of the disease, whether the agonist is administered for preventive or therapeutic purposes, previous therapy, the patient's clinical history and response to the agonist, and the discretion of the attending physician. The agonist is suitably administered to the patient at one time or over a series of treatments. In a combination therapy regimen, the compositions of the invention are administered in a therapeutically effective amount or a therapeutically synergistic amount. As used herein, a therapeutically effective amount is such that administration of a composition of the invention and/or co-administration of PLEXIND1 agonist and one or more other therapeutic agents, results in reduction or inhibition of the targeting disease or condition. The effect of the administration of a combination of agents can be additive. In some embodiments, the result of the administration is a synergistic effect. A therapeutically synergistic amount is that amount of PLEXIND1 agonist and one or more other therapeutic agents, e.g., an angiogenesis inhibitor, necessary to synergistically or significantly reduce or eliminate conditions or symptoms associated with a particular disease.

Depending on the type and severity of the disease, about 1 μg/kg to 50 mg/kg (e.g. 0.1-20 mg/kg) of PLEXIND1 agonist or angiogenesis inhibitor is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. Typically, the clinician will administered a molecule(s) until a dosage(s) is reached that provides the required biological effect. The progress of the therapy of the invention is easily monitored by conventional techniques and assays.

For example, preparation and dosing schedules for angiogenesis inhibitors, e.g., anti-VEGF antibodies, such as AVASTIN® (Genentech), may be used according to manufacturers' instructions or determined empirically by the skilled practitioner. In another example, preparation and dosing schedules for such chemotherapeutic agents may be used according to manufacturers' instructions or as determined empirically by the skilled practitioner. Preparation and dosing schedules for chemotherapy are also described in Chemotherapy Service Ed., M. C. Perry, Williams & Wilkins, Baltimore, Md. (1992).

Efficacy of the Treatment

The efficacy of the treatment of the invention can be measured by various endpoints commonly used in evaluating neoplastic or non-neoplastic disorders. For example, cancer treatments can be evaluated by, e.g., but not limited to, tumor regression, tumor weight or size shrinkage, time to progression, duration of survival, progression free survival, overall response rate, duration of response, and quality of life. Because the anti-angiogenic agents described herein target the tumor vasculature and not necessarily the neoplastic cells themselves, they represent a unique class of anticancer drugs, and therefore can require unique measures and definitions of clinical responses to drugs. For example, tumor shrinkage of greater than 50% in a 2-dimensional analysis is the standard cut-off for declaring a response. However, the PLEXIND1 agonists may cause inhibition of metastatic spread without shrinkage of the primary tumor, or may simply exert a tumoristatic effect. Accordingly, approaches to determining efficacy of the therapy can be employed, including for example, measurement of plasma or urinary markers of angiogenesis and measurement of response through radiological imaging.

The following examples are provided for illustrative purposes only and are not to be construed as limiting upon the teachings herein.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1

### Expression Constructs and Protein Purification

Human PlexinD1 ECD constructs (Met1 to Ala1271) were cloned into the eukaryotic expression vector pRK5 either as fusions to the Fc portion of human IgG1 (PlxD1-Fc) or to a C-terminal Histidine tag (PlxD1-His). Human Semaphorin 3E (Met1 to Ala554 of SEQ ID NO: 1) was cloned into a pRK5 vector with the Fc portion of human IgG1 as C-terminal fusion partner to generate the Sema3E^{SD}-Fc fusion construct. Human Semaphorin 3E (Ala26 to Arg560 of SEQ ID NO: 1) was cloned into a pRK5 vector with an N-terminal Histidine tag sequence to generate the Sema3E^{SD}-His fusion construct.

All proteins were produced by transient transfection of CHO cells. Proteins were purified to >95% purity by affinity chromatography using either protein-A Sepharose™ (GE Healthcare) for Fc fusion proteins or NiNTA Superflow (Qiagen) for Histidine tag fusions, followed by an ion exchange chromatography step (Q- or SP-Sepharose™, GE Healthcare). Size exclusion chromatography (Superdex™ 75 or 200, GE Healthcare) was used to further purify the protein if necessary. Protein identities were confirmed mass spectrometry analysis (LC-MS/MS after tryptic digest). Concentrations were determined by the BCA assay or by OD 280 absorption measurements. Purity and homogeneity were assessed by SDS-PAGE, size exclusion chromatography, and laser light scattering. Protein A levels for Fc tagged proteins as determined by protein A ELISA were below 50 ppm, and endotoxin levels as determined by the LAL (Limulus Amoebocyte Lysate) chromogenic endotoxin assay were below 0.5 EU/mg.

### Example 2

### Sema3E^{SD} Binds to PlexinD1

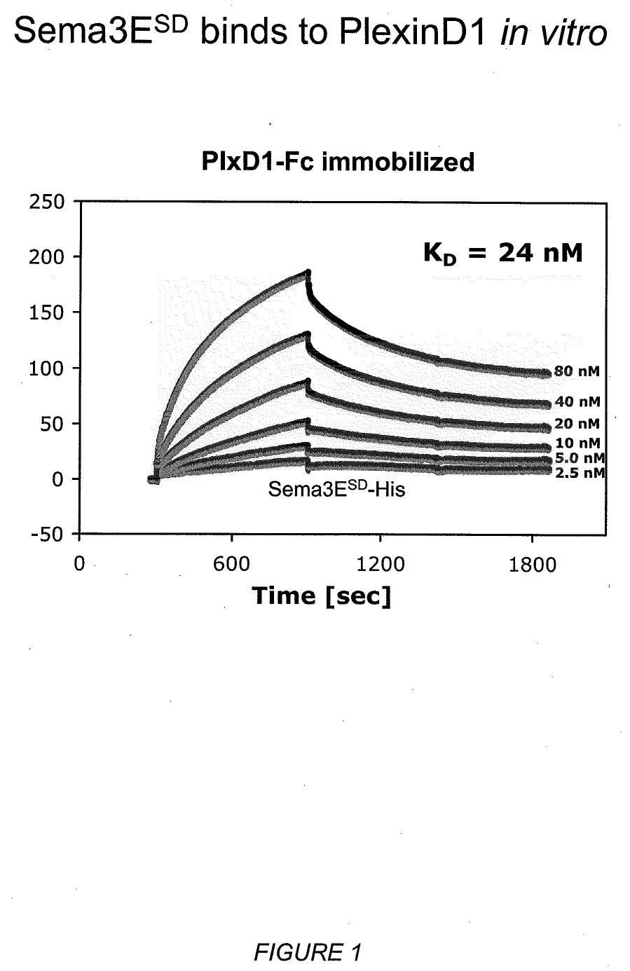

Binding experiments were performed by SPR measurements on a Biacore 3000 instrument (Biacore Inc.) at 25° C. PlxD1-Fc constructed as described in Example 1 was immobilized at high surface density (˜10,000 RU) on an activated CM5 chip using standard amine coupling procedures as described by the manufacturer. PlxD1-Fc was injected at a concentration of 10 μg/ml in 20 mM sodium acetate, pH 4.5 and at a flow rate of 5 μl/min until desired surface densities, measured in response units (RU), were reached. Unreacted groups were blocked by injecting 1 M ethanolamine. To perform binding assays, different concentrations of Sema3E^{SD}-His were injected in 10 mM HEPES pH 7.4, 150 mM NaCl, 0.005% v/v Surfactant P20, at a flow rate of 5 μl/min. Blank surfaces were used for background corrections. Injections of 10 mM glycine, pH 3.0 at 100 μl/min for 1 min were used to regenerate surfaces between two binding experiments. Kinetic analysis was performed with binding sensorgrams of different analyte concentrations using the BIAevaluation Software (Biacore Inc.). An one-to-one binding model was used to fit the experimental data for association and dissociation reactions. Dissociation constants were calculated from the on and off rates. As shown in FIG. 1, these experiments showed that Sema3E^{SD}-His bound to PlxD1-Fc with a K_{D} of 24 nM. Fluorescence-Activated Cell Sorting (FACS) analysis was used to demonstrate that Sema3E^{SD}-Fc binds both to mouse microvascular endothelial (MS1) cells, which express murine PlexinD1, and human umbilical vein endothelial cells (HUVECs), which express human PlexinD1. Radioligand cell binding assays were performed and the dissociation constant for Sema3E^{SD}-Fc binding to HUVECs was calculated to 4 nM.

### Example 3

### Sema3E^{SD} Inhibits HGF- and VEGF-Induced HUVEC Migration In Vitro and Does not Stimulate Migration by Itself

Migration assays were performed using a modified Boyden chamber with the 8 μm pore size Falcon™ 24-multiwell insert system (BD Biosciences). The plates were pre-coated with 8 μg/ml Laminin overnight at 37° C. Confluent HUVECs were starved overnight, harvested and resuspended in assay medium (EBM-2, 0.1% BSA). 100 μl cells with or without proteins as indicated were added into the upper chamber, whereas migration stimuli (HGF or VEGF, R&D Systems) were added to the lower chamber in 500 μl assay medium. Cells were allowed to migrate for 16 hrs at 37° C. To stop the assay, cells on the upper face of the membrane were removed with a sponge swab, and cells on the lower face were fixed with methanol and stained with SYTOX® Green (Molecular Probes). Images were taken with an inverted fluorescent microscope, and cell number was analyzed with ImageJ.

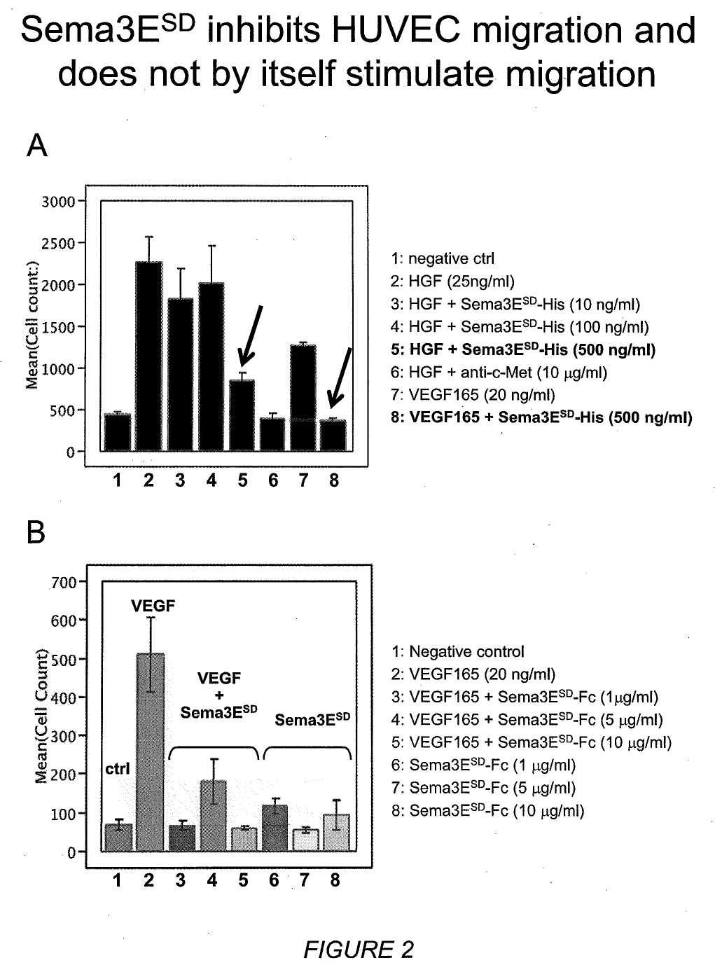

As shown in FIG. 2, the addition of HGF resulted in significant migration of HUVEC cells and Sema3E^{SD}-His inhibited the HGF activity in a concentration dependent manner (compare conditions 2 and 5; anti-c-Met antibody served as a positive control). Similarly, Sema3E^{SD}-His inhibited VEGF-induced HUVEC migration (compare conditions 7 and 8). These data demonstrate that Sema3E^{SD} inhibits both HGF- and VEGF-induced migration of HUVEC cells.

As shown in FIG. 2B, also the Sema3E^{SD}-Fc immunoadhesin inhibits VEGF induced HUVEC migration but does not have any migration stimulatory effect by itself.

### Example 4

### Sema3E^{SD} Inhibits HUVEC Sprout Formation In Vitro

Dextran-coated Cytodex 3® microcarrier beads (Amersham) were incubated with HUVEC cells (400 cells per bead) in EGM-2 overnight at 37° C. HUVEC coated beads were then washed three times with 5 ml clotting medium (EGM-2 minus VEGF), and resuspended in clotting medium with 2.5 μg/ml fibrinogen (Sigma) at a density of 200 beads/ml. To induce clotting, 0.5 ml fibrinogen/bead solution was added into one well of a 24-well tissue culture plate containing 0.625 units thrombin (Sigma), and incubated for 5 min at RT, then for 20 min at 37° C. The clot was equilibrated in 1 ml clotting medium for 30 min at 37° C. The medium was then replaced with 1 ml clotting medium containing skin fibroblast cells (Detroit 551, ˜30,000 cells/ml). VEGF (R&D Systems) alone or VEGF plus Sema3E^{SD}-His protein were added as indicated, and the assay was monitored for 8 days with change in medium every other day. Each condition was repeated in two wells. Images of the beads were captured by an inverted microscope, and concentric circles spaced at 100, 200, and 300 μm were drawn around the bead in each image. The number of vessels crossing each line was counted.

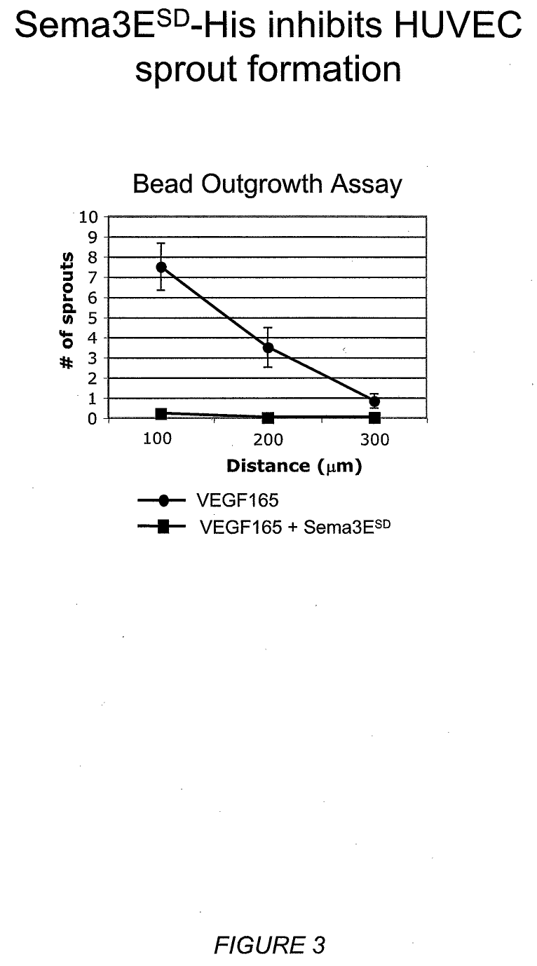

As shown in FIG. 3, the addition of Sema3E^{SD} significantly inhibited the ability of VEGF to induce HUVEC sprout formation.

### Example 5

### Sema3E^{SD}-Mediated Inhibition of HUVEC Migration Requires PlexinD1 Binding

Migration assays were performed using a modified Boyden chamber with the 8 μm pore size Falcon 24-multiwell insert system (BD Biosciences). The plates were pre-coated with 8 μg/ml Laminin overnight at 37° C. Confluent HUVECs were starved overnight, harvested and resuspended in assay medium (EBM-2, 0.1% BSA). 100 μl cells with or without proteins as indicated were added into the upper chamber, whereas migration stimuli (HGF, R&D Systems) were added to the lower chamber in 500 μl assay medium. In certain experiments, an anti Neuropilin antibody that blocks binding of class 3 semaphorins to Neuropilin 1 and 2 (panNrp^{A}; 10 μg/ml), PlexinD1 immunoadhesin (PlxD1-Fc; 9 ng/ml), antagonistic anti-plexinD1 antibodies (20 μg/ml), or anti-c-Met were added to the lower chamber at the concentrations indicated. Cells were allowed to migrate for 16 hrs at 37° C. To stop the assay, cells on the upper face of the membrane were removed with a sponge swab, and cells on the lower face were fixed with methanol and stained with SYTOX® Green (Molecular Probes). Images were taken with an inverted fluorescent microscope, and cell number was analyzed with ImageJ.

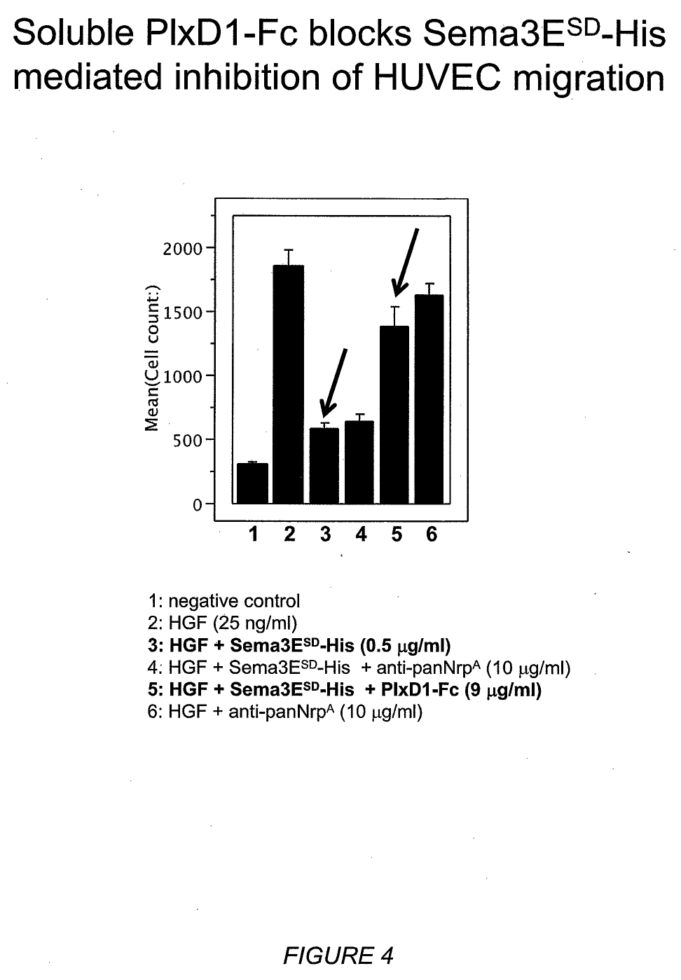

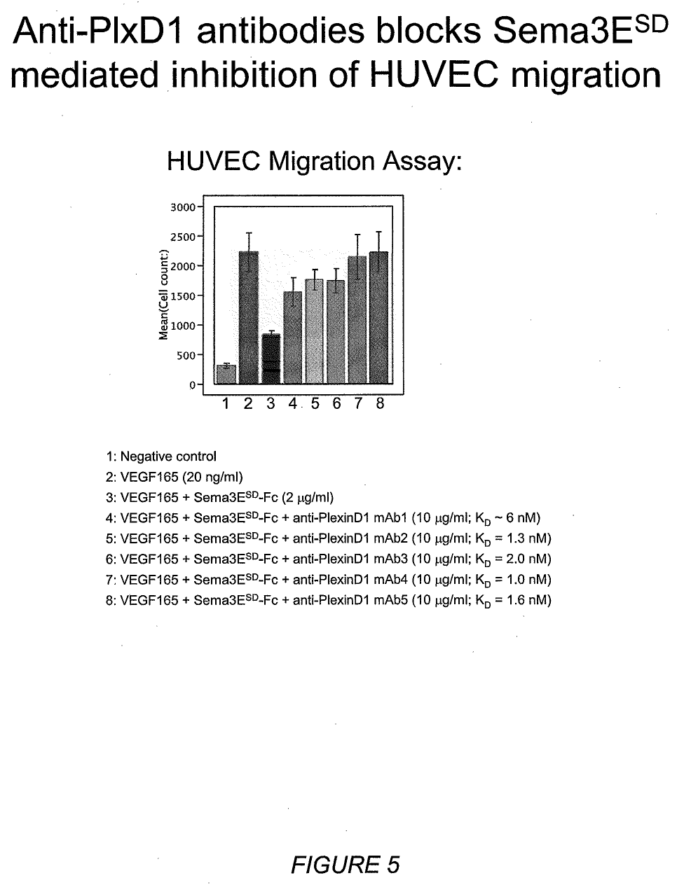

As shown in FIG. 4, the addition of PlxD1-Fc (condition 5) but not panNrpA (condition 4) blocks the ability of Sema3E^{SD}-His to inhibit HUVEC migration. Thus, the addition of a large excess of soluble PlexinD1, which inhibits binding of Sema3E^{SD}-His to native PlexinD1 on the cells, blocks Sema3E^{SD}-His activity. Similarly, blocking the ability of Sema3E^{SD}-His to interact with native PlexinD1 using anti-PlexinD1 antibodies blocks the ability of Sema3E^{SD}-His to inhibit HUVEC migration (FIG. 5). Strong and specific binding of anti-PlexinD1 antibodies to HUVECs was confirmed by Fluorescence-Activated Cell Sorting (FACS) analysis. These data confirm that Sema3E^{SD}-mediated inhibition of HUVEC activity requires interaction with PlexinD1 but not with Neuropilins.

### Example 6

### Sema3E^{SD} Inhibits Tumor Growth In Vivo

A suspension of 10⁷ Calu-6 tumor cells in Matrigel™ were injected sc in the back of Beige Nude female mice (Harlan Sprague). A total of 20 mice were injected and two days after cell injection tumor size was measured. Mice were divided into 2 equal groups with equal volumes of tumors and treated with either vehicle (PBS) or 10 mg/kg of Sema3E^{SD}-Fc every other day. Tumor growth was monitored by caliper measurements once per week for the first 2 weeks. After 2 weeks post cell injection tumors were measured at each dosing. The study was continued until day 41.

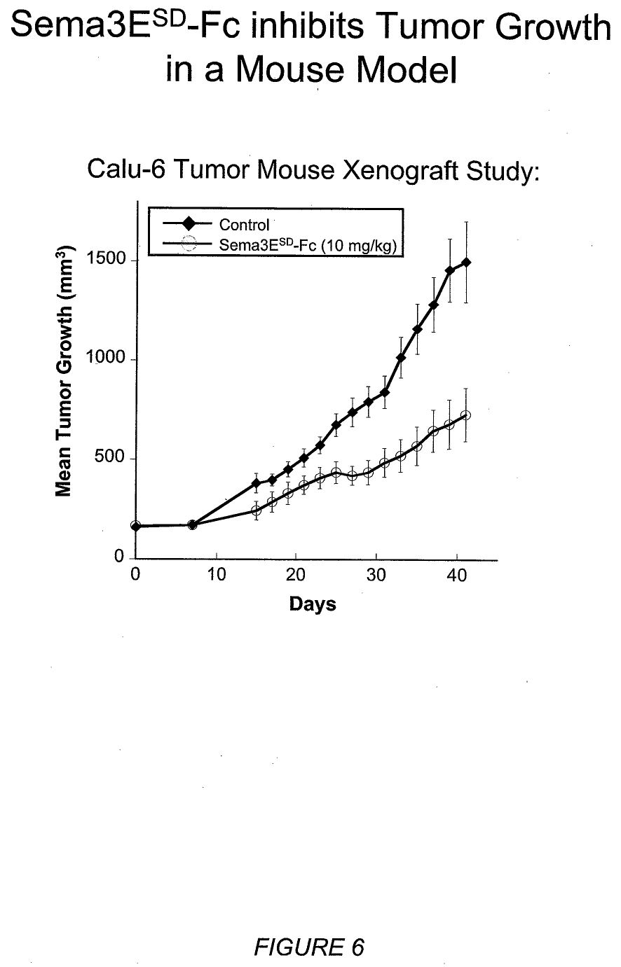

As shown in FIG. 6, treatment with Sema3E^{SD}-Fc significantly reduced tumor growth in this model.

What is claimed is:

1. A method for treating a proliferative disorder in an animal comprising administering to the animal a polypeptide comprising a fragment of Sema3E, wherein said polypeptide is a PlexinD1 agonist.

2. The method of claim 1, wherein the proliferative disorder is cancer.

3. The method of claim 2, wherein the cancer is carcinoma, lymphoma, blastoma, sarcoma, leukemia, squamous cell cancer, lung cancer, brain cancer, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, colorectal cancer, endometrial, uterine carcinoma, salivary gland carcinoma, renal cancer, prostate cancer, vulval cancer, thyroid cancer, hepatic carcinoma and cancers of the head and neck.

4. The method of any one of claims 1 to 3, wherein said fragment of Sema3E lacks the immunoglobulin-like domain.

5. The method of any one of claims 1 to 3, wherein said fragment of Sema3E lacks the first furin cleavage site.

6. The method of any one of claims 1 to 3, wherein said fragment of Sema3E does not bind to neuropilin-1 (Nrp1).

7. The method of any one of claims 1 to 6, further comprising administering a chemotherapeutic agent.

8. The method of any one of claims 1 to 7, further comprising administering a VEGF antagonist.

9. The method of claim 8, wherein said VEGF antagonist is selected from the group consisting of an antisense RNA, an siRNA, an aptamer, a polypeptide comprising a VEGF-binding fragment of a VEGF receptor, and an anti-VEGF antibody.

10. The method of claim 9, wherein said VEGF antagonist is an anti-VEGF antibody.

11. The method of claim 10, wherein said anti-VEGF antibody is Avastin®.

12. A polypeptide comprising a fragment of Sema3E, wherein said polypeptide is a PlexinD1 agonist and wherein said fragment lacks the first furin cleavage site.

13. The polypeptide of claim 12, wherein said fragment of Sema3E further lacks the immunoglobulin-like domain.

14. The polypeptide of claim 12 or 13, further comprising the Fc portion of an immunoglobulin.

15. The polypeptide of claim 14, wherein the immunoglobulin is human IgG1.

16. The polypeptide of claim 15, further comprising a linker between said fragment of Sema3E and said Fc portion of an immunoglobulin.

17. The polypeptide of claim 16, wherein said linker is selected from GRAG (SEQ ID NO: 2) and GGGS (SEQ ID NO: 3).

18. The polypeptide of any one of claims 12 to 17, wherein said fragment of Sema3E comprises from any one of amino acids 1-32 of SEQ ID NO: 1 to any one of amino acids 516-555 of SEQ ID NO: 1.

19. The polypeptide of claim 18, wherein said fragment comprises amino acids 1-554 of SEQ ID NO: 1 or amino acids 26-555 of SEQ ID NO: 1.

20. A nucleic acid encoding the polypeptide of any one of claims 12 to 19.

21. A vector comprising the nucleic acid of claim 20.

22. A host cell comprising the vector of claim 21.

23. A method for inhibiting angiogenesis in an animal comprising administering to the animal the polypeptide of any one of claims 12 to 19.

24. The method of claim 23, further comprising administering to the animal a second angiogenesis inhibitor.

25. The method of claim 24, wherein said second angiogenesis inhibitor is a VEGF antagonist.

26. The method of claim 25, wherein said VEGF antagonist is selected from the group consisting of an antisense RNA, an siRNA, an aptamer, a polypeptide comprising a VEGF-binding fragment of a VEGF receptor, and an anti-VEGF antibody.

27. The method of claim 26, wherein said VEGF antagonist is an anti-VEGF antibody.

28. The method of claim 27, wherein said anti-VEGF antibody is Avastin®.

29. A method for treating cancer in an animal comprising administering to the animal the polypeptide of any one of claims 12 to 19.

30. The method of claim 29, wherein the cancer is carcinoma, lymphoma, blastoma, sarcoma, leukemia, squamous cell cancer, lung cancer, brain cancer, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, colorectal cancer, endometrial, uterine carcinoma, salivary gland carcinoma, renal cancer, prostate cancer, vulval cancer, thyroid cancer, hepatic carcinoma and cancers of the head and neck.

31. The method of claim 29 or 30, further comprising administering a chemotherapeutic agent.

32. The method of any one of claims 29 to 31, further comprising administering a VEGF antagonist.

33. The method of claim 32, wherein said VEGF antagonist is selected from the group consisting of an antisense RNA, an siRNA, an aptamer, a polypeptide comprising a VEGF-binding fragment of a VEGF receptor, and an anti-VEGF antibody.

34. The method of claim 33, wherein said VEGF antagonist is an anti-VEGF antibody.

35. The method of claim 34, wherein said anti-VEGF antibody is Avastin®.
